(19) United States
(12) Patent Application Publication
(10) Pub. No.: US 2009/0142281 A1
(43) Pub. Date: Jun. 4, 2009

(54) Composition Comprising A Coupled Enzyme System

(76) Inventors: Thomas Rand, Brondby (DK); Susan Mampusti Madrid, Vedbaek (DK)

Correspondence Address: FROMMER LAWRENCE & HAUG, 745 FIFTH AVENUE- 10TH FL., NEW YORK, NY, 10151, US

(21) Appl. No.: 12/106,780

(22) Filed: Apr. 21, 2008

Related U.S. Application Data
(63) Continuation-in-part of application No. PCT/DK2006/000590, filed on Oct. 20, 2006.

(30) Foreign Application Priority Data
Oct. 21, 2005 (DK) PA200501474

(51) Int. Cl. A61K 8/66; A23L 1/28; C11D 3/386; C09D 189/00; A61Q 11/00; C11D 3/395; A61K 38/44

(52) U.S. Cl. 424/48; 426/61; 424/94.4; 424/50; 424/62; 510/392; 252/186.1; 106/124.1

(57) ABSTRACT

The present invention relates to a composition comprising a coupled enzyme system for the rapid and efficient production of hydrogen peroxide by the coupling of a first enzyme system capable of hydrogen peroxide generation, to a second enzyme system which utilizes the non hydrogen peroxide product of the first enzyme system, and optionally is capable of generating further hydrogen peroxide.

### REFERENCE TO RELATED APPLICATIONS

This application is a continuation-in-part of International Patent Application PCT/DK2006/000590 filed Oct. 20, 2006 and published as WO 2007/045251 on Apr. 26, 2007, which claims priority from Danish Application PA200501474 filed Oct. 21, 2005. Each of the above referenced applications, and each document cited in this text (“application cited documents”) and each document cited or referenced in each of the application cited documents, and any manufacturer's specifications or instructions for any products mentioned in this text and in any document incorporated into this text, are hereby incorporated herein by reference; and, technology in each of the documents incorporated herein by reference can be used in the practice of this invention.

It is noted that in this disclosure, terms such as “comprises”, “comprised”, “comprising”, “contains”, “containing” and the like can have the meaning attributed to them in U.S. patent law; e.g., they can mean “includes”, “included”, “including” and the like. Terms such as “consisting essentially of” and “consists essentially of” have the meaning attributed to them in U.S. patent law, e.g., they allow for the inclusion of additional ingredients or steps that do not detract from the novel or basic characteristics of the invention, i.e., they exclude additional unrecited ingredients or steps that detract from novel or basic characteristics of the invention, and they exclude ingredients or steps of the prior art, such as documents in the art that are cited herein or are incorporated by reference herein, especially as it is a goal of this document to define embodiments that are patentable, e.g., novel, nonobvious, inventive, over the prior art, e.g., over documents cited herein or incorporated by reference herein. And, the terms “consists of” and “consisting of” have the meaning ascribed to them in U.S. patent law; namely, that these terms are closed ended.

### FIELD OF THE INVENTION

The present invention relates to a composition comprising a coupled enzyme system for the rapid and efficient production of hydrogen peroxide by the coupling of a first enzyme system capable of hydrogen peroxide generation, to a second enzyme system which utilizes the non hydrogen peroxide product of the first enzyme system, and optionally is capable of generating further hydrogen peroxide.

### BACKGROUND OF THE INVENTION

Oral malodour and discoloration of the teeth are conditions that affect many people. Malodour of the oral cavity is also known as halitosis or bad breath. It is generally believed that the cause of this condition is due to the presence of anaerobic bacteria, especially gram-negative anaerobic bacteria, in the mouth. These bacteria will generate volatile sulphur compounds (VSC) which are known to cause breath malodour.

Dental plaque is a yellowish bio-film that builds up on the teeth. If not removed regularly, it can lead to dental cavities (caries), gingivitis and peridontitis and eventually tooth loss. The microorganisms that form the biofilm are almost entirely bacteria, with the composition varying by location in the mouth. Periodontal disease affects the periodontium, which is the investing and supporting tissues surrounding a tooth (i.e., the periodontal ligament, the gingiva, and the alveolar bone). Gingivitis and periodontitis are inflammatory disorders of the gingiva and the deeper periodontal tissues, respectively.

Today consumers are very interested in making their teeth whiter. People with whiter teeth are considered as having more personal confidence and better social acceptance. Teeth comprise both an inner dentin layer and an outer hard enamel layer. The enamel layer protects the inner dentin layer and live tissue and serves as the contact surface for mastication of solid food. The enamel layer is generally translucent and slightly off-white in colour. It is also considered porous since the hydroxy apatite crystals that comprise the enamel form microscopic hexagonal rods or prisms having microscopic pores or channels between them. As a result of this porous structure, staining agents and discolouring substances, such as antibiotics, foods containing colouring materials, coffee, cola, tea, tobacco, etc., can permeate the enamel and change its surface to appear yellow or brownish in colour.

While good oral hygiene, as achieved by brushing the teeth with a cleansing dentifrice, may help reduce the incidence of stain, gingivitis, plaque, periodontal disease, and/or breath malodour, it does not necessarily prevent or eliminate their occurrence. Microorganisms contribute to both the initiation and progression of gingivitis, plaque, periodontal disease, and/or breath malodour. Thus, in order to prevent or treat these conditions, these microorganisms must be suppressed by some means other than simple mechanical scrubbing. In addition, simple mechanical scrubbing will not be entirely effective to remove all stain types and/or whiten the teeth.

Enzymes which belong to EC class 1.1.3. are oxidoreductases which utilise oxygen as acceptor, and CH—OH groups are the donor. The capability of such oxygen oxidoreductases to generate hydrogen peroxide, which has an antimicrobial effect, has been utilized to improve the storage stability of certain food products including cheese, butter and fruit juice as it is disclosed in JP-B-73/016612. It has also been suggested that oxidoreductases may be potentially useful as oxygen scavengers or antioxidants in food products. Tooth bleaching composition comprising oxidoreductase(s) is described in U.S. Pat. No. 6,379,653, where bleaching of teeth was obtained by treatment with glucose oxidase. Glucose oxidase is highly specific for glucose and requires presence of this cariogenic sugar that degrades in the mouth to compounds responsible for cavities.

WO97/06775 discloses oral compositions which comprise at least one oxidoreductase. The oxidoreductases considered by WO97/06775 include enzymes within the enzyme classes comprising oxidases including E.C. 1.1.3. E.C. 1.2.3, E.C. 1.3.3, E.C. 1.4.3, E.C. 1.5.3, E.C. 1.7.3, E.C. 1.8.3, E.C. 1.9.3, laccases and related enzymes comprised in E.C. 1.10.3 and peroxidases in E.C. 1.11. Substrates that are not cariogenic, such as amino acids, alcohol, sugar alcohol, such as xylitol and sorbitol are considered as suitable substrates for oxidoreductases. A specific xylitol oxidase considered is the xylitol oxidase disclosed in JP 80892242, which is reported to oxidize xylitol, D-sorbitol, D-galactitol, D-mannitol and D-arabinitol in the presence of oxygen.

The inclusion of certain oxidative enzymes in oral compositions such as toothpastes, mouthrinses and dentifrices can reduce plaque and gingivitis. The enzymes that have been used include as their active ingredients, amyloglucosidase and glucose oxidase. These produce hydrogen peroxide from dietary fermentable carbohydrates which in turn converts thiocyanate to hypothiocyanite in the presence of salivary lactoperoxidase. The resultant hypothiocyanite acts as a bacterial inhibitor by interfering with cell metabolism. Sorbitol oxidase is known e.g. from Hiraga K. et al. “Molecular cloning and expression of a gene encoding a novel sorbitol oxidase from Streptomyces sp. H-7775.”; Biosci. Biotechnol. Biochem. 62:347-353 (1998) describing cloning and expression of sorbitol oxidase from Streotomyces sp.

The majority of sugar substitutes approved for food use are artificially synthesized compounds. However, some natural sugar substitutes are known—including sorbitol and xylitol, which are found in berries, fruit, vegetables and mushrooms. Although natural, they may be produced synthetically in bulk food production, to lower production costs. Both xylitol and sorbitol are used in oral care compositions such as toothpaste or chewing gum to give a sweet taste and, in the case of xylitol, for decreasing lactic acid production and increasing saliva production (Hayes C. J Dent Educ. 65(10): 1106-1109 2001).

Even though a great deal of research has been carried out in order to find compositions useful for the treatment and/or prevention of gingivitis, plaque, periodontal disease, and/or breath malodor and/or for the whitening of teeth, additional efficacious compositions and methods of treatment for these purposes are still desirable.

Detergents for laundry and dish washing consist of complex mixtures of a wide variety of ingredients, which typically include a number of components such as ionic and non-ionic surfactants, solvents, builders, perfumes, enzymes, and bleaching components. In such complex mixtures, storage stability problems, particularly of enzymes, are well known. In some cases, stability problems are related to the physical stability of the detergent, while in other cases, it relates to the functional stability of the individual ingredients in the detergent. Bleaching agents such as percarbonates and perborates, are commonly used in powder detergents where they, together with bleach activators (e.g., tetra acetylethylenediamine (TAED) and nonanoyloxybenzenesulfonate (NOBS)), act to generate peracids (e.g. peracetic acid), hydrogen peroxide, and/or other related species upon addition of water during the wash cycle. The peracids or the other active oxygen species then act to bleach or lighten certain stains on the fabric or dishware. However, there is no ideal bleaching system available for use in aqueous liquid formulations. In addition, there is a need for the production of bleaching agents (e.g., active oxygen species, peroxide, and peracids) upon dilution of the detergent in the laundry wash liquor to bleach and/or lighten stains.

Enzymes such as oxidases are in particular susceptible to storage stability issues in liquid detergent formulation. This prevents their widespread use in fabric and house hold cleaning compositions that involve bleaching action. Maintaining the oxidase enzymatic activity in detergents during storage has been a challenge, especially in detergents that also contain oxidase substrate components. The presence of both oxidase and oxidase substrate results in the in situ generation of peroxygen. This results in decreased enzyme stability due to oxidation of the enzymes both in liquid and dry formulations. Peroxides damage enzymes by various mechanisms such as oxidizing some of the amino acid residues in the enzyme or by interacting with the enzymes' cofactors. This often results in a gradual loss of activity. In dry detergent formulations enzymes can be stabilized by (e.g. encapsulation of the enzymes as described in WO 96/02623.

Citation or identification of any document in this application is not an admission that such document is available as prior art to the present invention.

### SUMMARY OF THE INVENTION

The present invention relates to a composition comprising a first enzyme, a substrate for the first enzyme, and a further enzyme, and the use thereof for whitening and/or bleaching of e.g. teeth, skin, hair, textiles or paper, an oral care product, and a method for whitening and/or bleaching of teeth, the use as a preservative and as anti-microbial agent, use in cosmetics, in detergents, in paints, in food and feed, in food and feed production and preparation, and in pesticides.

The present invention is based upon a surprising synergy which the present inventors have found when a hydrogen peroxide generation system comprising a first enzyme, such as a polyol oxidase, and a first substrate, such as a polyol, is coupled to a further enzyme system, such as an oxidoreductase enzyme system which utilizes the non hydrogen peroxide product generated by the first oxidase (i.e. the second substrate) optionally generating further hydrogen peroxide.

The coupling of the first and second enzyme (systems) has been found to greatly enhance the efficiency of the hydrogen peroxide production from the first enzyme system, and can also result in production of hydrogen peroxide from both the first and second substrates. The effect is a considerably higher hydrogen peroxide generation and due to the surprising synergy between the first oxidase and the further enzyme/oxidoreductase, a far higher rate of hydrogen peroxide compared to what would have been expected from the first substrate/first oxidase enzyme system alone (or the further oxidoreductase enzyme system alone). It is as if the coupling of the further enzyme system ‘turbo-charges’ the first oxidase, forcing the very high level of hydrogen peroxide production.

The first enzyme is preferably an oxidase, and is referred to as a first oxidase herein. A preferred oxidase is polyol oxidase, such as sorbitol oxidase.

The present invention provides detergent compositions comprising the composition, of the invention as well as methods for the use of the composition of the invention in liquid detergent compositions for bleaching and cleaning, for example of coloured food stains.

During the development of the present invention, it was surprisingly found that sorbitol oxidases were suitable for use in bleaching systems that avoided the disadvantages plaguing currently used bleaching systems.

The coupling of the first oxidase to a further enzyme therefore allows full exploitation of the oxidative capacity locked up in the first substrate. The first oxidase appears to act as a ‘key’ which is surprisingly robust in a detergent environment, and, as disclosed herein, allows efficient and rapid production of hydrogen peroxide bleaching power, especially when coupled to a further oxidoreductase.

The present invention provides in one aspect a composition comprising a first oxidase, a first substrate, and an oxidoreductase, wherein the first substrate is oxidisable by the first oxidase to form hydrogen peroxide and a second substrate, and the second substrate is oxidisable by the oxidoreductase to form hydrogen peroxide and a product.

In a further aspect the invention provides an oral care product comprising a composition according to the invention and ingredients used in oral care products.

In a further aspect the invention provides a cosmetic product comprising a composition according to the invention and one or more ingredients used in cosmetic products.

In a further aspect the invention provides a detergent product comprising a composition according to the invention and or more ingredients used in detergent products.

In a further aspect the invention provides a paint product comprising a composition according to the invention and or more ingredients used in paint products.

In a further aspect the invention provides a pesticide product comprising a composition according to the invention and or more ingredients used in pesticide products.

In a further embodiment, the invention provides for a bleaching or whitening product, including but not limited to a bleaching or whitening product for bleaching or whitening external mammalian tissue, including but not limited to skin, hair or teeth comprising a composition according to the invention and ingredients used in bleaching and whitening products suitable for application on external mammalian tissue.

In a further embodiment, the invention provides for a cosmetic method for bleaching or whitening of external mammalian tissue comprising contacting the external mammalian tissue with a composition according to the invention or the product for bleaching and/or whitening external mammalian tissue according to the invention in an amount and duration suitable for bleaching and/or whitening the external mammalian tissue.

The invention also provides for a medicament comprising a composition according to the invention.

The invention also provides for an edible beverage comprising a composition according to the invention, such as a fruit juice.

In one embodiment, the composition does not comprise the first substrate, but the first substrate is either naturally present or is added to the composition or application matrix.

The invention further provides for a detergent or bleaching product comprising a composition according to the invention and at least one further ingredient used in detergent or bleaching products.

The invention further provides for the use of the composition according to the invention in an oral care product with beneficial teeth bleaching and/or whitening effects, and/or extended shelf life, and/or anti-microbial/anti-bacterial effects either prior to or during use.

The invention further provides for the use of a composition according to the invention in an edible product with beneficial prebiotic effects when consumed by an individual mammal, and/or a prolonged shelf life.

The invention further provides for the use of a composition according to the invention in a cosmetic product which has a prolonged shelf life, and/or is capable of bleaching and/or whitening external mammalian tissue, and/or has an anti-microbial/anti-bacterial effect when applied to the human skin.

The invention further provides for the use of a composition according to the invention in a paint product which shows improved preservation either before or after application, and/or shows reduced anti-fouling.

The invention further provides for a method for the preparation of a composition comprising admixing a first enzyme and a first substrate, and at least one further enzyme, wherein the first substrate is oxidisable by the first enzyme, including but not limited to the sorbitol oxidase, to form hydrogen peroxide and a second substrate, and the second substrate is convertable by the at least one further enzyme to form a product.

The composition may comprise a suitable matrix component or components to which the first enzyme and at least one further enzyme are admixed. The first substrate may also be admixed into the matrix component, or, in one embodiment form part or even the whole of the matrix component. The matrix component may therefore consist or comprise of the first substrate.

The invention further provides for a method for the preparation of a composition comprising admixing a first oxidase and a first substrate, and at least one further oxidoreductase, wherein the first substrate is oxidisable by the first oxidase, including but not limited to the sorbitol oxidase, to form hydrogen peroxide and a second substrate, and the second substrate is oxidisable by the oxidoreductase to form hydrogen peroxide and a product.

The invention further provides for the use of a composition according to the invention, in the manufacture of a medicament for the treatment or prevention of a medical disorder selected from: gum disease, gingivitis, periodontal disease, irritable bowel syndrome, lactose intolerance, colon cancer, high blood cholesterol, high blood pressure, hypertension, infection, inflammation and nutritional deficiencies.

The invention further provides for a method of medical treatment comprising administering the composition according to the invention, or medicament or oral care products according to the invention to a patient in need of treatment or prophylaxis.

The invention further provides for a method of generating hydrogen peroxide, the method comprising admixing a first enzyme and a first substrate, and at least one further enzyme under conditions suitable for the generation of hydrogen peroxide from the oxidation of the first substrate due to the activity of the first enzyme, and optionally generation of further hydrogen peroxide from the oxidation of a second substrate due to the activity of the at least one further enzyme, wherein the second substrate is generated by the oxidation of the first substrate by the first oxidase, and wherein the second substrate is converted into a product by the at least one further enzyme.

The invention further provides for a method of generating hydrogen peroxide, the method comprising admixing a first oxidase and a first substrate, and at least one further oxidoreductase under conditions suitable for the generation of hydrogen peroxide from both the oxidation of the first substrate due to the activity of the first oxidase, and the generation of hydrogen peroxide from the oxidation from a second substrate due to the activity of the at least one further oxidoreductase, wherein the second substrate is generated by the oxidation of the first substrate by the first oxidase.

In another aspect the invention provides the use of the composition according to the invention for whitening and/or bleaching.

In another aspect the invention provides a method for bleaching and/or whitening of teeth, comprising contacting the teeth with an oral care product comprising a composition according to the invention in an amount and time suitable for bleaching and/or whitening teeth.

The invention provides for the use of a composition according to the invention for whitening and/or bleaching teeth.

Although the main aspects of the invention refer to a coupled enzyme system, in some embodiments, including but not limited to the following embodiments, the invention provides for compositions which comprise a polyol oxidase and a first substrate, as referred to herein. The use of the polyol oxidase/first substrate enzyme system has been found to be highly beneficial in these applications, including but not limited to the generation of hydrogen peroxide from a non fermentable substrate, optionally without lowering the pH (e.g. like when not coupled to a further oxidoreductase system), and the anti-microbial/bacterial, anti-spoilage, bleaching and whitening characteristics thereby provided.

The invention provides for a paint composition comprising a polyol oxidase and a first substrate, wherein the first substrate is oxidisable by the polyol oxidase to form hydrogen peroxide.

The invention provides for a cosmetic composition comprising a polyol oxidase and a first substrate, wherein the first substrate is oxidisable by the polyol oxidase to form hydrogen peroxide.

The invention provides for a food or feed composition comprising a polyol oxidase and a first substrate, wherein the first substrate is oxidisable by the polyol oxidase to form hydrogen peroxide, including but not limited to a food or feed composition that is selected from the group consisting of: Dairy products, including but not limited to milk, cream, cheese, whey; beverages, including but not limited to fruit juice,

The invention provides for a medicament composition comprising a polyol oxidase and a first substrate, wherein the first substrate is oxidisable by the polyol oxidase to form hydrogen peroxide, including but not limited to when the first substrate is sorbitol or preferably xylitol.

The invention provides for a pesticide composition comprising a polyol oxidase and a first substrate, wherein the first substrate is oxidisable by the polyol oxidase to form hydrogen peroxides.

These and other embodiments are disclosed or are obvious from and encompassed by, the following Detailed Description.

### BRIEF DESCRIPTION OF THE DRAWINGS

The following detailed description, given by way of example, but not intended to limit the invention solely to the specific embodiments described, may best be understood in conjunction with the accompanying drawings, in which:

FIG. 1: The expression plasmid (pKB105-TAT-Sox-7775).

FIG. 2: The plasmid “pKB105-CelA-Sox7775”.

FIG. 3: The initial velocity of H₂O₂ production using the compositions with between 1× and 3000× excess of the further enzyme (oxidoreductase) compared to the first oxidase (SOX), as measured over 5 minutes in 300 uL ABTS assay. A dramatic synergy was seen with both the glucose oxidase and hexose oxidase further enzymes, with upto about 250-300% increase in hydrogen peroxide production rate seen.

FIG. 4: The initial velocity of H₂O₂ production using the compositions with between 1× and 3000× excess of the further enzyme (oxidoreductase) compared to the polyol oxidase, as measured over 5 minutes in 300 μL ABTS assay. A dramatic synergy was seen with both the glucose oxidase and hexose oxidase further enzymes, especially at dosages greater than 1×, such as at least 2× dosage of the further enzymes compared to the polyol oxidase.

FIG. 5a: pET 24a—sorbitol oxidase (H7775) expression vector.

FIG. 5b. Expression of active Sorbitol oxidase in E. coli BL21(DE3) pLysS strain:

a) Expression vector containing the 1.27 NdeI-BamHI fragment, encoding the Streptomyces H-7775 SOX synthetic gene.

b) In gel overlay activity assay (PMS/NBT) using sorbitol as substrate. Negative control: Glucose oxidase (GOX) in lane 1, lanes 2-10 are cell lysates from the different transformants.

FIG. 6. The construct for expression of the putative SOX gene in Streptomyces lividans strain g3s3. The putative SOX gene was cloned as NcoI-BamH1 PCR fragment and inserted.

### DETAILED DESCRIPTION OF THE INVENTION

The present invention relates in one aspect to a composition comprising a first enzyme including but not limited to a polyol oxidase (e.g. sorbitol oxidase), a first substrate, and a further enzyme including but not limited to a oxidoreductase, wherein the first substrate is oxidisable by the first oxidase, to form hydrogen peroxide and a second substrate, and the second substrate is convertible by the further enzyme to produce a product.

Preferably the further enzyme is a further oxidoreductase, and the second substrate is oxidisable by the oxidoreductase to generate hydrogen peroxide and the (further) product.

The composition according to the invention is applicable for all purposes where production of H₂O₂ is needed e.g. in applications where bleaching and/or whitening is required or for antimicrobial purposes, and especially in products where non-toxic or environmentally acceptable ingredients are desired.

### The First Substrate

In the present context the term “first substrate” refers to a substrate which is oxidisable by first enzyme (such as the first oxidase, such as sorbitol oxidase) to generate hydrogen peroxide and a second substrate.

In one aspect of the invention the first substrate is a polyol, including but not limited to one or more substrates selected from sugar alcohols including but not limited to those selected from the group consisting of D-sorbitol, D-xylitol, D-mannitol, D-arabitol, glycerol, inositol, 1,3-propanediol, 1,3-butanediol, and 1,4-butanediol.

In one embodiment, the first substrate is one or more polyols selected from the group consisting of D-sorbitol or D-xylitol.

In one embodiment the first substrate is D-sorbitol.

It is recognized that sugars and sugar-alcohols that exist in D or L stereoisomers, it is the D form which is prevalent in nature, and are therefore preferred in terms of the first and second substrates as referred to herein.

In a further aspect of the invention the first substrate is a non-cariogenic sweetener including but not limited to those selected from the group consisting of D-sorbitol or D-xylitol. In yet a further aspect the first substrate is D-sorbitol. D-sorbitol and D-xylitol are essentially non-cariogenic and are already used in oral care products e.g. in chewing gum as an artificial sweetener with beneficial results (Hayes C. J Dent Educ. 65(10): 1106-1109 2001).

In one aspect the first substrate is one or more sugar alcohol substrates selected from the group consisting of sorbitol, xylitol, maltitol, mannitol, galactitol, isomalt, lactitol, arabitol, and erythritol.

In one aspect the first substrate may be selected from the group consisting of: ribitol, threitol, lyxitol, allitol, altritol, gulitol, iditol, talitol, pentitol and hexitol.

In one aspect the first substrate is one or more sugar alcohol substrates selected from the group consisting of sorbitol, xylitol, maltitol, mannitol, galactitol, isomalt, lactitol, arabitol, erythritol, glycerol, inositol, 1,2-propanediol, 1,3-butanediol, and 1,4-butanediol.

In a preferred aspect of the invention the first substrate is or comprises sorbitol.

In one aspect of the invention the first substrate is or comprises xylitol.

The polyol oxidase and further oxidoreductase are oxidases which are capable of generating peroxide (H₂0₂).

The level of polyol present in the composition according to the invention will depend upon the application and the formulation used. For use in oral care products a high level of polyol may be used, where the polyol may be the major matrix ingredient in the composition. Polyols can also form a major component of cosmetic formulations. In such applications polyols may be added as humectants. Polyols may also be added to detergents including but not limited to soaps, where they can also have a humectant function or as a clarifying agent.

However, in some applications, such as in some paint and detergent applications the polyol may be added as a minor component, sufficient to provide enough first substrate for the generation of hydrogen peroxide, but not forming a major matrix component.

Therefore, for example, the level of first substrate present in the composition of the invention, prior to the oxidation into the second substrate may be between about 0.05% to about 80% w/w, including but not limited to between 0.1% and about 70% w/w.

Suitably, in one embodiment, the level of polyol present in oral care products may therefore be between about 1 to about 80% w/w, including but not limited to between about 10 to about 75% w/w, or including but not limited to between about 20 to about 70% w/w.

Suitably, in one embodiment, the level of polyol present in paint products may range from between about 0.01 to about 20% w/w, including but not limited to from about 0.1 to about 10% w/w, including but not limited to from about 1 to about 5% w/w.

Suitably, in one embodiment, the level of polyol present in a cosmetic composition or products according to the invention may range from between about 1 to about 50% w/w, including but not limited to between about 5 to about 40% w/w, or including but not limited to between about 10 to about 40% w/w. U.S. Pat. No. 7,094,395 discloses cosmetics which comprise about 8-32% polyol (humectants), such a range may also be used in the compositions of the present invention.

Suitably, in further embodiments, the level of polyol present in detergent products may range from between about 0.01% to about 40% w/w, including but not limited to between about 0.1% to about 30%, including but not limited to between about 1% to about 20%, including but not limited to between about 1% to about 10% or between about 1% and about 5%.

### The First Enzyme

The first enzyme is typically an oxidase enzyme, and is referred to as ‘first oxidase’ herein.

The first enzyme, such as first oxidase may be derived or isolated from an organism including but not limited to those selected from the group consisting of: Streptomyces, Xanthomonas, Brevibacterium, Frankia, Nocardia, Janibacter, Burkholderia, Paracoccus, Chromabacterium, Thermobifida, Pseudomonas, Corynebacterium and Bacillus species and their homologs

Suitable first oxidases may include enzymes which are categorized under an Enzyme Classification number (E.C.) selected from the group consisting of: EC 1.1.3.14 catechol oxidase, EC 1.1.3.18 secondary-alcohol oxidase, EC 1.1.3.41 xylitol oxidase, EC 1.1.3.13 alcohol oxidase, EC 1.1.3.194-hydroxymandelate oxidase, EC 1.1.3.20 longchain alcohol oxidase, EC 1.1.3.40 D-mannitol oxidase. EC 1.1.3.7 arylalcohol oxidase, EC 1.1.3.30 polyvinylalcohol oxidase, EC 1.1.3.21 glycerolalcoholoxidase, and EC 1.1.3.38 vannilylalcohol oxidase.

JP 80892242 discloses a xylitol oxidase which oxidises xylitol, D-sorbitol, D-galactitol, D-mannitol and D-arabinitol in the presence of oxygen.

A xylitol oxidase can be obtained from strains of Streptomyces sp. (e.g. Streptomyces IKD472, FERM P14339) having a pH optimum at 7.5, is stable at pH 5.5 to 10.5 and at temperatures up to 65° C.; properties very well suited for the applications disclosed herein, including but not limited to oral care and detergent compositions and products.

In one specific embodiment, the first enzyme is not the xylitol oxidase which can be obtained from strains of Streptomyces sp. (e.g. Streptomyces IKD472, FERM P14339) having a pH optimum at 7.5, and which is stable at pH 5.5 to 10.5 and at temperatures up to 65° C.

During the development of the present invention, it was surprisingly found that polyol oxidases such as sorbitol oxidases were suitable for use in bleaching systems that avoided the disadvantages plaguing currently used bleaching systems. These sorbitol oxidases include enzymes isolated from such organisms as Streptomyces or Xanthomonas species and their homologs. However, it is not intended that the present invention be limited to these specific nor any particular sorbitol oxidase(s).

Sorbitol oxidase (“SOX” or “SoX”) is an enzyme that catalyzes conversion of sorbitol to glucose and hydrogen peroxide. Sorbitol oxidases are known and used in various applications (See e.g., Oda and Hiraga, Ann. NY Acad. Sci., 864:454-457 [1998]; and Yamashita et al J. Biosci. Bioengin., 89:350-360 [2000]). Sorbitol (D-glucitol, C₆H₁₄O₆, MW 182.2, CAS 50-70-4) is a commonly used in enzyme product formulations. Thus, sorbitol oxidase provides an attractive biobleaching agent for use in detergents that incorporate these sorbitol-containing enzyme product formulations.

In one embodiment, the first oxidase has a higher specific activity on sorbitol as compared to xylitol, including but not limited to at least about 1.5×, or at least about 2×, higher specific activity on sorbitol as compared to xylitol

In one embodiment, the first oxidase has a specific activity on sorbitol of at least about 5 units/mg.

The specific activity of the first oxidase on sorbitol and xylitol substrates may be determined in vitro, including but not limited to using the assays provided in the examples, or alternatively the specific activity may be determined in situ, within said oral care composition.

A preferred SOX is an oxidoreductase that uses covalently bound FAD as a cofactor for oxidation of sorbitol to glucose. This enzyme offers a unique opportunity for its potential use as a biobleach agent on its own, as well as used in combination with carbohydrate oxidases including but not limited to glucose oxidase and/or hexose oxidase (see WO 96/39851), (gluco)oligosaccharide oxidase and M. nivale carbohydrate oxidase (see WO99/31990).

An advantage of the use of such combinations is due to the fact that SOX converts sorbitol to glucose, which can then be converted to gluconate by glucose oxidase and/or hexose oxidase, thus generating two moles of hydrogen peroxide per mole of sorbitol, as illustrated below.

D-Sorbitol+O₂→D-Glucose+H₂O₂

D-Glucose+O₂→D-Gluconate+H₂O₂

A preferred glycerol oxidase (GLOX) is an enzyme found in the genera Penicillium and Botrytis (See e.g., Lin et al Enz. Micro. Technol., 18:383-387 [1996]; and Uwajima et al, Agric. Biol. Chem., 44:399-406 [1989]). This enzyme catalyzes the conversion of glycerol and oxygen to glyceraldehyde and hydrogen peroxide as shown below.

CH₂OH—CHOH—CH₂OH+O₂→CH₂OH—CHOH—CHO+H₂O₂

Glycerol (glycerin, C₃H₈O₃, MW 92.09, CAS 56-81-5) is commonly used in enzyme product formulations, soap and detergent formulations, food and beverages, pharmaceuticals and is widely used in cosmetics and personal care applications. Thus, glycerol oxidase provides an attractive biobleaching agent for use in detergents that incorporate these glycerol-containing enzyme product formulations.

During the development of the present invention, sorbitol oxidase was isolated from Streptomyces lividans (SCO6147) (SEQ ID NO 2) and Streptomyces sp. H7775 (SEQ ID NO 1) (See, Hiraga et al., Biosci. Biotech. Biochem., 61:1699-1704 [1997]). The sorbitol oxidase was expressed both intracellularly and extracellularly from these organisms. The prosthetic group is a covalently bound FAD (1 mol of FAD to 1 mol of SOX). Thus, it is a flavoprotein, with typical absorption maxima at 276, 358, and 455 nm for the H7775 SOX, 345 nm for the SCO₆₁₄₇ SOX (as expressed in S. lividans), which is indicative of a histidine-flavin linkage. Flavin is functionally involved in oxidation of sorbitol as observed by desired changes in UV-VIS spectra. FAD is very tightly bound with the protein and thus offers a stable enzyme for laundry applications.

The SOX gene was cloned and sequenced from Streptomyces species H-7775 (Genbank accession number AB000519).

The sorbitol oxidase gene from Streptomyces species H-7775 (Genbank accession number AB000519) comprises a 1260 bp open reading frame (ORF) encoding a protein having 420 amino acids with theoretical MW of 45,158 Daltons. The enzyme is stable for 24 hours at 30° C., between pH 7.5-10 with an optimum temperature of 50° C. at pH 7.5. It is also heat stable up to 55° C. The nearest homolog identified for this enzyme is xylitol oxidase (51% homology). SOX is an efficient enzyme for multiple applications, including detergents, fabric care, home care, oral care (e.g., dental whitening and/or cleaning), personal care, textile processing, food processing and industrial cleaning. In addition, numerous SOXs can catalyze other substrates including but not limited to xylitol, mannitol, arabitol, ribitol, erythritol, inositol, glycerol, propane diol, and butane diol. Thus, this enzyme uses a wider spectrum of substrates, providing flexibility in substrate usage in various applications.

Many of these first substrates, as provided herein, are present in typical detergent, oral care and cosmetic formulations or can be added to them.

The amino acid sequence of sorbitol oxidase from Streptomyces sp. H7775 is known in the art and set forth in SEQ ID NO:1.

As indicated above, the polyol oxidase such as the sorbitol oxidases utilised herein were found to be thermally stable and stable over a wide pH range. Indeed, the pH profile of the sorbitol oxidase used were found to be compatible with the pHs necessarily used in industry, as well as detergents and other cleaning agents.

In addition, the polyol oxidase such as the sorbitol oxidase provided by the present invention may preferably produce sugar including but not limited to glucose, i.e. an aldehyde product that can be further oxidized to gluconic acid, a carboxylic acid product, using other oxidases including but not limited to hexose oxidase or glucose oxidase releasing another molecule of hydrogen peroxide from starting substrate sorbitol. Similarly oxidation of polyols including but not limited to xylitol, arabitol, mannitol, by sorbitol oxidase, xylitol oxidase, mannitol oxidase with the assistance of atmospheric oxygen with formation of the corresponding sugar, including but not limited to xylose, arabinose, mannose, respectively as secondary substrate for further oxidation by other relevant oxidases including but not limited to hexose oxidase, xylose oxidase, pyranose oxidase, arabinose oxidase, and mannose oxidase is feasible.

For use in edible and oral care compositions, it is also advantageous to use enzymes being substantially active at pHs prevailing in the mouth, i.e. between pH 5.0 to 9.0, preferably between pH 6.0 to 8.5, especially between pH 6.4 to 7.5.

In one embodiment, the polyol oxidase is a pentitol or hexitol oxidase.

In one embodiment the polyol oxidase is not a triose oxidase, or is not a glycerol oxidase.

It is recognised that enzymes may have activity on more than one substrate. Therefore, when we refer to an enzyme by the name of the compound which it oxidises, for example sorbitol oxidase (sorbitol) or hexose oxidase (hexose) or glucose oxidase (glucose), the name refers to either the classification which the enzyme has been given, such as the EC number, or the name the enzyme is referred to in the art, or the predominant activity as compared to the substrate for which the enzyme has the highest specific activity. In this respect, a sorbitol oxidase has a higher specific activity on sorbitol than, for instance xylitol. Suitably, sorbitol oxidase may, in one embodiment, also catalyse the oxidation of several polyols including at least two of the group consisting of D-sorbitol, D-xylitol, D-mannitol, D-arabitol, glycerol, inositol, 1,2-propanediol, 1,3-butanediol, and 1,4-butanediol.

In one embodiment the polyol oxidase is selected from the group consisting of ribitol oxidase, threitol oxidase, xylitol oxidase, allitol oxidase, altritol oxidase, gulitol oxidase, iditol oxidase, talitol oxidase, pentitol oxidase and hexitol oxidase.

In one embodiment the polyol oxidase has a ratio of specific activity on the polyol it is named after, compared to the specific activity on an alternative substrate, as listed herein as either said first substrate or said second substrate, excluding the substrate the polyol is named after, of greater than 1, such as greater than about 1.5, such as greater than about 2, such as greater than about 3, such as greater than about 4, such as greater than about 5, such as greater than about 10. In one embodiment the polyol oxidase has a ratio of specific activity on sorbitol compared to the specific activity on an alternative substrate, of greater than 1, such as greater than about 1.5, such as greater than about 2, such as greater than about 3, such as greater than about 4, such as greater than about 5, such as greater than about 10, wherein the alternative substrate is selected from the group consisting of: a sugar such as maltose, hexose, glucose, mannose, galactose, isomaltulose, lactose, arabinose, erythrose, pentose, xylose and triose, preferably glucose; a triose polyol, such as glycerol; and xylitol.

In one embodiment the polyol oxidase is not xylitol oxidase.

In one embodiment, the first oxidase, such as the polyol oxidase exhibits a higher activity on sorbitol than xylitol, such as at least one and a half times as much activity, such as at least two times as much activity. In the same embodiment or in a different embodiment, the polyol oxidase has no more than three times the activity on sorbitol as compared to xylitol.

In one embodiment, the first oxidase, such as the polyol oxidase is selected from the group consisting of: sorbitol oxidase, xylitol oxidase, maltitol oxidase, mannitol oxidase, galactitol oxidase, isomalt oxidase, lactitol oxidase, arabitol oxidase, arabitol oxidase and erythritol oxidase.

In one embodiment, the first oxidase, such as the polyol oxidase has a (specific) oxidase activity of at least about 5 units/g protein when using the respective (e.g. polyol) substrate, including but not limited to a substrate selected from the group consisting of: Sorbitol, xylitol, maltitol, mannitol, galactitol, isomalt, lactitol, arabitol, arabitol and erythritol.

A preferred first oxidase, such as the polyol oxidase is sorbitol oxidase, or an enzyme which exhibits sorbitol activity.

In one embodiment the first oxidase, such as the polyol oxidase is xylitol oxidase, or exhibits xylitol oxidase activity.

In one embodiment the first oxidase, such as the polyol oxidase is a glycerol oxidase, or comprises glycerol oxidase activity.

In one embodiment, the first oxidase, such as the polyol oxidase has a ratio of specific activity on the respective polyol to the corresponding sugar of greater than 1, such as at least about 1.5, such as at least about 2, such as at least about 3, such as at least about 4, such as at least about 5, such as at least about 10. In one embodiment, the first oxidase, such as the polyol oxidase has a ratio of specific activity on the respective polyol to glucose of greater than about 1, such as at least about 1.5, such as at least about 2, such as at least about 3, such as at least about 4, such as at least about 5, such as at least about 10.

In one embodiment, the first oxidase, such as the polyol oxidase, including but not limited to the sorbitol oxidase or xylitol oxidase is derived or obtained from a strain of Streptomyces, including but not limited to Streptomyces coelicolor, or Streptomyces sp. IKD472.

In one embodiment, the first oxidase, such as the polyol oxidase, such as the sorbitol oxidase is derived from or obtained from a strain of Streptomyces coelicolor.

In a preferred embodiment, first oxidase, such as the polyol oxidase, such as the sorbitol oxidase, is a polypeptide consisting of, or derived from SEQ ID NO 1 or SEQ ID NO 2 and homologues, variants or fragments thereof.

In one embodiment, using the assay provided by Example 6, the polyol oxidase has activity on D-sorbitol, D-xylitol, D-mannitol, D-ribitol, myo-inositol, and glycerol. In a further embodiment, which may be the same or different, the polyol oxidase has activity on 1,3 propanediol and/or 1,2 propanediol.

In one embodiment, using the assay provided by Example 6, the polyol oxidase does not have activity on propylene glycol and/or ethylene glycol.

Sorbitol oxidase (SOX) may be obtained from suitable microorganisms such as Streptomyces. Hiragi K. et al. (Biosci. Biotechnol. Biochem. 62:347-353 (1998)) describes production of recombinant sorbitol oxidase in E. coli. Other sources are described in e.g. U.S. Pat. No. 5,741,687 and U.S. Pat. No. 5,472,862 wherein a microorganism from the soil of Sekigahara-cho, Fuwa-gun, Gifu Prefecture, Japan, from the genus Xanthomonas, such as Xanthomonas maltophilia TE3539 (FERM BP-4512) is described. Other examples of polyol oxidases are mannitol oxidase (EC 1.1.3.40) from the snails Helix aspersa and Arion ater that catalyzes the oxidation of D-arabinitol, D-mannitol and, to a lesser extent, D-glucitol (sorbitol), and xylitol oxidase (EC 1.1.3.41) from Streptomyces coelicolor that oxidises D-xylitol to xylose and H₂O₂ and D-sorbitol to glucose and H₂O₂.

The first oxidase, such as the polyol oxidase, such as a sorbitol or xylitol oxidase may be obtained from a microbial source such as a bacterium or a fungus. In particular from bacteria classified into the class Actinobacteria and order Actinomycetales.

The first oxidase, such as the polyol oxidase, including but not limited to a sorbitol or xylitol oxidase may be obtained from different species of Streptomyces, Xanthomonas, Brevibacterium, Frankia, Nocardia, Janibacter, Burkholderia, Paracoccus, Chromabacterium, Thermobifida, Pseudomonas, Corynebacterium and Bacillus species and their homologs.

The first oxidase, such as the polyol oxidase, such as a sorbitol or xylitol oxidase may be obtained from a strain of Streptomyces, preferably from a strain of Streptomyces coelicolor or Streptomyces sp. IKD472 (Yamashita, Mitsuo et al., Journal of Bioscience and Bioengineering (2000), 89(4), 350-360), and Streptomyces sp. H-7775. (Hiraga, Kazumi et al., Bioscience, Biotechnology, and Biochemistry (1997), 61(10), 1699-1704).

JP 09206072 discloses a sorbitol oxidase of Streptomyces and its production method and use, which may also be used in the present invention.

JP 06169764 discloses a sorbitol oxidase of Xanthomonas which may also be used in the present invention.

In one aspect of the invention the first oxidase, such as the polyol oxidase, such as the sorbitol oxidase is derived from a strain of Streptomyces.

In a further aspect the first enzyme, such as the first oxidase, such as the polyol oxidase e.g. the sorbitol oxidase is produced by recombinant methods.

In one embodiment, first enzyme/oxidase and the further enzyme are active at ambient temperature.

In one embodiment, first enzyme/oxidase and the further enzyme are active at mouth or body temperature, such as about 37° C.

Although it is recognised that the skilled person will dose the first enzyme/oxidase at a level suitable (an effective amount) for the required purpose, it is envisaged that in one embodiment the first oxidase is at a level of between about 0.1 and about 200,000 units per kg, such as between about 0.1 and about 100,000 units per kg, such as between about 1 and about 100,000 units per kg, such as between about 5 and about 50,000 units per kg, such as between about 10 and about 20,000 units per kg, such as between about 100 and about 10,000 units per kg. Other suitable ranges, for use in some embodiment, include between about 10 and about 1,000 units per kg, or between about 1 and about 10,000 units per kg.

In one embodiment, referring to an oral care composition between 1 and 10000 U (units)/100 g first oxidase may be used. Dosages around 5-20 units per 100 g are also considered appropriate for some embodiments.

For use in food and feed compositions, the above dosage ranges may also be appropriate.

In one embodiment, referring to a paint composition between about 10 and about 10000 U/100 g first oxidase such as the polyol oxidase may be used. Dosages around 10-50 units per 100 g are also considered appropriate for some embodiments.

In one embodiment, referring to a cosmetic composition between about 1 and about 10000 U/100 g first oxidase such as the polyol oxidase may be used. Dosages around 5-20 units per 100 g are also considered appropriate for some embodiments.

In one embodiment, referring to detergent composition between about 0.1 and about 10000 U/100 g first oxidase such as the polyol oxidase may be used. Dosages around 5-100 units per 100 g are also considered appropriate for some embodiments.

### The Second Substrate

The second substrate is capable of being converted by the (at least one) further enzyme to the product. The second substrate is, preferably oxidisable by the further enzyme (further oxidoreductase) to generate further hydrogen peroxide and the product.

In the present context the term “second substrate” refers to a substrate which is a result of the oxidation of the first substrate and is convertable by the further enzyme to form the product.

In a preferable embodiment, the term “second substrate” refers to a substrate which is a result of the oxidation of the first substrate by the first enzyme and is oxidisable by the further oxidoreductase to form hydrogen peroxide and the product.

The second substrate is, in one aspect, one or more sugars, such as sugars selected from the group consisting of glucose, xylose, maltose, mannose, galactose, isomaltulose, lactose, arabinose and erythrose.

The second substrate is, in one aspect, one or more sugars, including but not limited to sugars selected from the group consisting of ribose, lyxose, allose, altrose, gulose, idose, and talose.

The second substrate may therefore be, or comprise glucose, for example when the first substrate is or comprises sorbitol.

Due to the remarkable synergy between the first oxidase, such as the polyol oxidase and the further enzyme, such as the further oxidoreductase, the steady state level of the second substrate is typically low. In oral care products, as well as preservative applications, for example in paint or cosmetics, the low level of the second substrate may be highly advantageous, particularly when the second substrate is a fermentable or cariogenic sugar, i.e. compounds which can encourage or ‘feed’ the growth of detrimental organisms including but not limited to micro-organisms/bacteria (e.g. cariogenic bacterial in the oral cavity), or algae and barnacles (fouling on maritime paint for example). Therefore, not only does the invention provide a highly efficient and voluminous production of hydrogen peroxide, it can also provide a system where there is a minimal level of fermentable substrates, reducing the likelihood of undesirable growth of detrimental organisms.

In one embodiment, the level of the second substrate, as generated by the first oxidase, such as one or more fermentable or cariogenic sugars, present in the composition according to the invention is less than about 50%, such as less than about 25%, such as less than about 10%, such as less than about 5%, such as less than about 1%, such as less than about 0.5%, such as less than about 0.1%, such as less than about 0.05%, such as less than about 0.01% of the (initial) level of polyol present in the composition, as measured by a molar ratio.

In a preferred embodiment the second substrate is a sugar.

In one embodiment the sugar is a monosaccharide hexose, including but not limited to a hexose selected from the group consisting of glucose, fructose, galactose, mannose, sorbose.

In one embodiment the sugar is a monosaccharide pentose, including but not limited to a pentose selected from the group consisting of ribose, arabinose, xylose or lyxose.

In one embodiment the sugar is a triose, including but not limited to either aldotriose or ketotriose.

In one embodiment the sugar is a disaccharide including but not limited to sucrose or maltose.

### The Further Enzyme

The (at least one) further enzyme is characterised in that it is capable of converting the second substrate to produce a product.

The further enzyme is not the same enzyme, or enzymatic entity, as the first enzyme. However, in one embodiment, the further enzyme may be attached to the first enzyme, for example the first and further enzymes may be co-expressed as a polyprotein.

The further enzyme may be any enzyme which, within the composition according to the invention, or in the applications disclosed herein, is capable of converting the second substrate into a product, the presence of which does not detrimentally affect the generation of hydrogen peroxide from the oxidation of the first substrate by the first enzyme.

Examples of suitable further enzymes include glucose dehydrogenase (E.C: 1.1.1.118) which, when supplied with NAD+, results in the production of D-glucono-1,5-lactone+NADH; glucose 1-dehydrogenase (E.C: 1.1.1.119) (NADP(+)), which, when supplied with NADP+, results in the production of D-glucono-1,5-lactone+NADPH; glucokinase (E.C. 2.7.1.2, which phosphorylates the COOH group of glucose to produce glucose-6-phosphate; glucokinase, which converts D-glucose+D-fructose<=>D-gluconolactone+D-glucitol.

Therefore in one embodiment, the composition may comprise further compounds which are utilised by the further enzyme with the second substrate to produce the product(s)—suitably such further compound may, for example, be selected from the group consisting of NAD+, NADP+, or fructose.

In a preferred aspect the (at least one) further enzyme is a (at least one) further oxidoreductase (also referred to as oxidoreductase).

The further oxidoreductase is preferably an oxidase other than a polyol oxidase. The further oxidoreductase is not the polyol oxidase referred to herein within the context of the first oxidase.

Oxidoreductases are enzymes belonging to EC class 1.x.x.x. In the present invention the oxidoreductases utilise an oxygen acceptor, including but not limited to CH—OH or an aldehyde or oxo (EC 1.2.3.x).

In a preferred embodiment, oxidoreductases in the present context are enzymes belonging to EC class 1.1.3. acting with oxygen as acceptor. For example the hexose oxidase (D-hexose:O₂-oxidoreductase, EC 1.1.3.5) is an enzyme which in the presence of oxygen is capable of oxidizing D-glucose and several other reducing sugars including maltose, lactose and cellobiose to their corresponding lactones with subsequent hydrolysis to the respective aldobionic acids upon formation of hydrogen peroxide. The oxidation catalyzed by hexose oxidase on glucose and galactose can e.g. be illustrated as follows:

D-Glucose+O₂→δ-D-gluconolactone+H₂O₂, or

D-Galactose+O₂→γ-D-galactogalactone+H₂O₂

In another aspect of the invention the oxidoreductase is one or more selected from the group consisting of hexose oxidase, glucose oxidase, carbohydrate oxidase, and oligosaccharide oxidase such as (gluco)oligosaccharide oxidase. In a further aspect of the invention the oxidoreductase is one or more enzymes that catalyse oxidation of sugars including but not limited to those selected from the group consisting of a carbohydrate oxidase, (gluco)oligosaccharide oxidase, pyranose oxidase a hexose oxidase or a glucose oxidase. In a further aspect of the invention the oxidoreductase is a glucose oxidase and/or a hexose oxidase. In yet a further aspect of the invention the oxidoreductase is a hexose oxidase.

In a preferred embodiment, the oxidoreductase is a sugar oxidase, including but not limited to a sugar-oxidase selected from the group consisting of: carbohydrate oxidase, oligosaccharide oxidase, maltose oxidase, hexose oxidase, glucose oxidase, mannose oxidase, galactose oxidase, isomaltulose oxidase, lactose oxidase, arabinose oxidase, erythrose oxidase, pentose oxidase, xylose oxidase, triose oxidase,

In one embodiment, the sugar-oxidase is selected from the group consisting of: EC 1.1.3.4 glucose oxidase, EC 1.1.3.5 hexose oxidase, EC 1.1.3.9 galactose oxidase, EC 1.1.3.10 pyranose oxidase, EC 1.1.3.11 L-sorbose oxidase, and EC 1.1.3.40 D-mannitol oxidase.

Suitable oxidases may be identified under the Enzyme Classification number E.C. 1 (Oxidoreductases) in accordance with the recommendations (1992) of the International Union of Biochemistry and Molecular Biology (IUBMB)) which are enzymes catalysing oxidoreductions, in particular oxidases listed under E.C. 1.1.3. or E.C. 1.2.3, i.e. oxidases acts on molecular oxygen (O₂) and yield peroxide (H₂O₂), utilising either CH—OH or an aldehyde or oxo group as a donor.

A suitable glucose oxidase may originate from Aspergillus sp., including but not limited to a strain of Aspergillus niger, or from a strain of Cladosporium sp. in particular Cladosporium oxysporum, especially Cl. oxysporum CBS 163 described in WO 95/29996 (from Novo Nordisk A/S).

Hexose oxidases from the red sea-weed Chondrus crispus (commonly known as Irish moss) (Sullivan and Ikawa, (1973), Biochim. Biophys. Acts, 309, p. 11-22; Ikawa, (1982), Meth. In Enzymol. 89, carbohydrate metabolism part D, 145-149) oxidises a broad spectrum of carbohydrates, including but not limited to D-glucose, D-galactose, maltose, cellobiose, lactose, D-glucose 6-phosphate, D-mannose, 2-deoxy-D-glucose, 2-deoxy-D-galactose, D-fucase, D-glucuronic acid, and D-xylose.

Also the red sea-weed Iridophycus flaccidum produces easily extractable hexose oxidases, which oxidise several different mono- and disaccharides (Bean and Hassid, (1956), J. Biol. Chem., 218, p. 425; Rand et al. (1972, J. of Food Science 37, p. 698-710).

The broad substrate spectrum of hexose oxidase is advantageous in the connection with tooth bleaching as the total amount of usable substrate (i.e. carbohydrate) present in the mouth is significantly greater than for related enzymes having more specific catalytic properties.

Carbohydrate oxidase form Microdochium nivale described in EP 1041890 acts on several sugars, including glucose, lactose and xylose as well as on oligosaccharides.

Another oxidoreductase capable of acting on several sugars and oligosaccharides is obtained from Acremonium strictum (Lin, et al. Biochemica and Biophysica Acta 118 (1991)). Its use in bakery applications is described in JP 11056219.

Other examples of suitable oxidoreductases are glucose oxidase (EC 1.1.3.4) and galactose oxidase (EC 1.1.3.9).

In one embodiment the oxidoreductase is one or more selected from the group consisting of a carbohydrate oxidase, a hexose oxidase or a glucose oxidase.

In one embodiment the oxidoreductase is a hexose oxidase.

Another example of a suitable oxidoreductase is hexose oxidase (HOX) (EC 1.1.3.5) which may be obtained by isolating the enzyme from several red algal species including but not limited to Iridophycus flaccidum (Bean and Hassid, 1956) and Chondrus crispus (Sullivan et al. 1973). In a preferred aspect of the invention HOX is obtained or prepared as described in WO 01/38544. HOX is available from Danisco A/S as DairyHOX™

The dosage of the further oxidoreductase may be within the same ranges as referred to for the polyol oxidase above, although it is recognised that in one embodiment an excess of the further oxidase is added, as referred herein.

### The Enzyme Composition

It is preferred that purified enzymes, such as the sorbitol oxidase and/or further oxidoreductase are used, i.e. the enzymes are purified prior to being added to the composition of the invention. Enzyme purity is preferably determined using SDS-PAGE and densitometry. A purified enzyme is at least about 20% pure, such as at least about 30% pure, such as at least about 40% pure, such as at least about 50% pure. It is recognized that a purified enzyme may however be formulated with other proteins, for example mixed with protein stabilizers such as BSA or other enzymes, the assessment of enzyme purify therefore excludes proteins added to the enzyme after purification.

The rate of hydrogen peroxide production can be controlled by, for example, preparing a composition comprising specified ratio of the effective amount of the first enzyme/oxidase compared to the further enzyme/oxidoreductase. Typically, and as shown in the examples, an increasing excess of the further enzyme/oxidoreductase results in increased rate of hydrogen peroxide production.

In one embodiment the molar quantity of total hydrogen peroxide produced (or producible), is greater than the molar quantity of the first substrate converted (or convertible) to the second substrate or the amount of the first substrate present. Suitably, the molar quantity of hydrogen peroxide produced (or producible), is, in one embodiment, between greater than 100% and less than or equal to 200% the molar quantity of the first substrate converted (or convertible) to the second substrate, or the amount of the first substrate present. The use of the present invention may therefore allow up to two molecules of hydrogen peroxide to be produced from a sorbitol first substrate, as compared to a single molecule of hydrogen peroxide from the use of a sorbitol oxidase enzyme alone. In one embodiment, the molar quantity of hydrogen peroxide produced (or producible) is up to two times the molar quantity of hydrogen peroxide which can be produced from a polyol oxidase system without a further oxidoreductase.

In one preferred embodiment, the amount of the at the least one further enzyme/oxidoreductase compared to the amount of the first enzyme/oxidase, such as the polyol oxidase (e.g. sorbitol oxidase) present in the composition according to the invention is greater than 1, as measured by the respective number of enzyme units present in said composition, such as greater than about 1.5, such as greater than about 2, such as greater than about 3, such as greater than about 5, such as greater than about 10, such as greater than about 20, such as greater than about 50, such as greater than about 100, such as greater than about 150, such as greater than about 200, such as greater than about 300, such as greater than about 500, such as greater than about 1000.

A further advantage of the composition according to the invention is the possibility of choosing a combination of enzymes and substrates generating the whitening agent hydrogen peroxide without accumulating cariogenic sweeteners or sugars as a product.

As an example SOX catalyses the oxidation of D-sorbitol to yield 1 mole of D-glucose and 1 mole of H₂O₂. The oxidoreductase, such as HOX, catalyses the oxidation of D-glucose to yield 1 mole of D-gluconic acid and 1 mole of H₂O₂. Combined use of SOX and HOX thus generates 2 moles of H₂O₂ from 1 mole of sorbitol oxidase substrate as illustrated in the following scheme.

The result of the enzymatic reaction of SOX is the intermediate glucose, which is cariogenic, but has a short life-span as it is fast converted to gluconic acid. A further advantage of the present invention is that only one mole of gluconic acid is formed for every two moles of hydrogen peroxide.

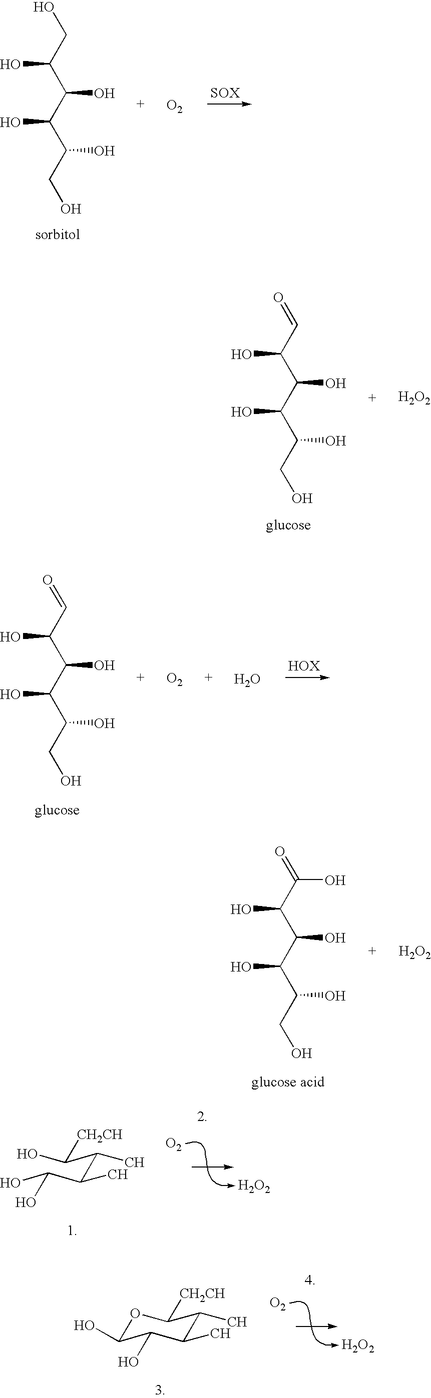

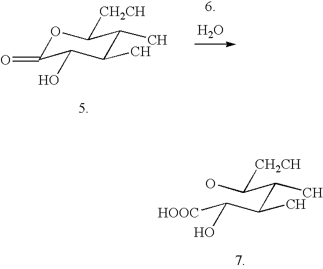

The reaction presented above represents the conversion of a polyol to the corresponding acid, by two consecutive enzymatic steps. Exemplified is the conversion of D-sorbitol to gluconic acid. (1) D-sorbitol, (2) Catalysis by polyol oxidase (3) D-glucose (4) Catalysis by hexose oxidase, glucose oxidase, glucooligosaccharide oxidase, carbohydrate oxidase (5) D-glucono-1,5-lactone (6) Hydrolysis in aqueous environment (7) D-gluconic acid.

The reaction may be generalised as:

| | |
|---|---|
| First Oxidase | Further Oxidoreductase |

| | | | | |
|---|---|---|---|---|
| Polyol | → | sugar | → | lactone |

Further specific examples include:

| | | | | |
|---|---|---|---|---|
| Sorbitol | → | glucose | → | D-glucono-1,5-lactone |
| Galactitol | → | galactose | → | D-galactono-1,5-lactone* |
| Mannitol | → | Mannose | | D-mannono-1,5-lactone |
| Lactitol | → | lactose | → | D-lactonono-1,5-lactone |
| Xylitol | → | xylose | → | D-xylono-1,5-lactone |

*(or D-galactohexadialdose (if galactose oxidase is used) oxidises at C6)

The lactone product is typically converted to an acid in an aqueous environment. The first oxidase may be selected from those disclosed herein. The further oxidoreductase enzyme is a sugar oxidase as referred to herein, including but not limited to hexose oxidase or mannose oxidase (for mannose).

The amount of each enzyme present in the composition of the invention, or the total amount of enzyme present, or added to the composition or (application) products as referred to herein will depend on the enzymes used and the desired formulation required, but typically may range from about 0.0001% to about 20%, such as about 0.001% to about 10%, such as about 0.005% to about 2%, such as about 0.01% to about 1% by weight of the final composition.

Typically, the coupling of the two enzymes has been found to give approximately 200-300% the rate of production of hydrogen peroxide compared to a first enzyme/first substrate system alone. Although considerable improvements in hydrogen peroxide were obtained using an equivalent unit to unit dose of both the polyol oxidase (such as sorbitol oxidase), and the further oxidoreductase such as hexose or glucose oxidase, the synergy was further enhanced by adding an excess of the further oxidoreductase to the composition, resulting in more hydrogen peroxide produced and at a faster rate.

In one aspect of the invention the composition comprises a sorbitol oxidase and a hexose oxidase and as the first substrate D-sorbitol.

In another aspect of the invention the composition comprises a sorbitol oxidase and a glucose oxidase and as the first substrate D-sorbitol.

In one embodiment, such as when the further oxidoreductase is HOX, the polyol oxidase, such as sorbitol or xylitol oxidase may catalyse the oxidation of D-xylitol to yield 1 mole of xylose and 1 mole of H₂O₂. This may be advantageous particularly in embodiment where there is an alternative polyol other than xylitol, such as sorbitol, in that due to the low activity of HOX on xylose, xylose will accumulate. Xylose is a prebiotic compound. Hence such compositions can be used in food and feed compositions, or in a medicament, or in the preparation of food or feed composition or medicament, to enhance the xylose content, whilst also obtaining the other beneficial effects of the invention.

The use of polyol oxidase, such as sorbitol oxidase or xylitol oxidase, and a polyol, such as D-xylitol or sorbitol, has the advantage that an anti-microbial and/or whitening effect by H₂O₂ is obtained using non-toxic ingredients, and the addition of cariogenic compounds such as sugar is avoided.

### The Composition Matrix

The composition according to the invention may, suitably, comprise other materials typically used in the products and applications referred to herein, or other suitable applications/products.

The matrix materials are suitably selected to ensure compatibility with the enzymes used in the composition to allow an effective amount of the enzymes to be used.

### The Product

As described above, a preferred embodiment is when the action of the further enzyme on the second substrate generates further hydrogen peroxide and a product. The product is therefore other than hydrogen peroxide.

In one embodiment, the product obtained by the action of the further enzyme on the second substrate may be more than a single product, i.e. may be products.

The accumulation of the product in the composition of the invention, or in the applications described herein, preferably does not negatively affect the production rate of hydrogen peroxide from the conversion of the first substrate by the first enzyme. Indeed, as the present inventors have discovered, the conversion of the second substrate to the product effectively removes the second substrate which not only greatly enhances the rate of generation of hydrogen peroxide by affecting the activity of the first enzyme on the first substrate, but also reduces the accumulation of possible undesirable second substrate.

In a preferred embodiment, the product is produced by the oxidation of the second substrate by the further oxidoreductase, and may for example be a lactone (oxidation at C1), a dialdose (oxidation at C5 for pentoses, C6 for hexose etc). One example is D-galactohexadialdose produced by galactose oxidase or a dehydro-sugar if oxidation occurs at any other position in the middle of the sugar chain. A further example is 2-dehydro-D-glucose produced by pyranose oxidase by oxidation of glucose at C2.

The product may be selected from the group consisting of D-glucono-1,5-lactone, D-xylono-1,5-lactone, D-maltono-1,5-lactone, D-mannono-1,5-lactone, D-galactono-1,5-lactone, D-hexadialdose, D-lactono-1,5-lactone, D-arabono-1,5-lactone, D-erythrono-1,5-lactone, D-ribono-1,5-lactone, D-lyxono-1,5-lactone, D-allono-1,5-lactone, D-altrono-1,5-lactone, D-gulono-1,5-lactone, D-idono-1,5-lactone, D-talono-1,5-lactone, and the lactone of isomaltulose, and possibly some more products from the oxidation of galactose oxidase on the C6 position of sugars.

### DEFINITIONS

Unless otherwise indicated, the practice of the present invention involves conventional techniques commonly used in molecular biology, microbiology, protein purification, protein engineering, protein and DNA sequencing, recombinant DNA fields, and industrial enzyme use and development, all of which are within the skill of the art.

Unless defined otherwise herein, all technical and scientific terms used herein have the same meaning as commonly understood by one of ordinary skill in the art to which this invention pertains. For example, Singleton and Sainsbury, Dictionary of Microbiology and Molecular Biology, 2d Ed., John Wiley and Sons, NY (1994); and Hale and Margham, The Harper Collins Dictionary of Biology, Harper Perennial, N.Y. (1991) provide those of skill in the art with a general dictionaries of many of the terms used in the invention. Although any methods and materials similar or equivalent to those described herein find use in the practice of the present invention, preferred methods and materials are described herein. Accordingly, the terms defined immediately below are more fully described by reference to the Specification as a whole. Also, as used herein, the singular terms “a,” “an,” and “the” include the plural reference unless the context clearly indicates otherwise. Unless otherwise indicated, nucleic acids are written left to right in 5′ to 3′ orientation; amino acid sequences are written left to right in amino to carboxy orientation, respectively. It is to be understood that this invention is not limited to the particular methodology, protocols, and reagents described, as these may vary, depending upon the context they are used by those of skill in the art.

It is intended that every maximum numerical limitation given throughout this specification includes every lower numerical limitation, as if such lower numerical limitations were expressly written herein. Every minimum numerical limitation given throughout this specification will include every higher numerical limitation, as if such higher numerical limitations were expressly written herein. Every numerical range given throughout this specification will include every narrower numerical range that falls within such broader numerical range, as if such narrower numerical ranges were all expressly written herein.

When referring to a “respective polyol substrate” of an enzyme, it refers to the substrate which the enzyme utilises, e.g. the respective substrate of sorbitol oxidase is sorbitol, and the respective substrate of xylitol oxidase is xylitol.

As used herein, the term “compatible,” means that the composition matrix materials (other ingredients) do not reduce the enzymatic activity of the oxidase enzyme(s) provided herein to such an extent that the oxidases(s) is/are not effective as desired during normal use situations. Specific cleaning composition materials are exemplified in detail hereinafter.

As used herein, “effective amount of enzyme” refers to the quantity of enzyme necessary to achieve the enzymatic activity required in the specific application. Such effective amounts are readily ascertained by one of ordinary skill in the art and are based on many factors, including but not limited to the particular enzyme variant used, the cleaning application, the specific composition of the cleaning composition, and whether a liquid or dry (e.g., granular) composition is required, and the like.

Amino acid and polynucleotide homology may be determined using ClustalW algorithm using standard settings including but not limited to those described in the align program available at web pages maintained by the European Bioinformatics Institute website: Method: EMBOSS::water (local): Gap Open=10.0, Gap extend=0.5, using Blosum 62 (protein), or DNAfull for nucleotide sequences.

It is preferable the when referring to “the second substrate is convertable by the at least one further enzyme to form a product”, that the second substrate is oxidisable by the further enzyme (oxidoreductase) to form hydrogen peroxide and a (the) product.

The term “variant(s)” as used herein in the context of a polypeptide (sequence), such as SEQ ID NO 1 and SEQ ID NO 2 refers to a polypeptide which is prepared from the original (parent) polypeptide, or using the sequence information from the polypeptide, by insertion, deletion or substitution of one or more amino acids in said sequence, i.e. at least one amino acids, but preferably less than about 50 amino acids, such as less than about 40, less than about 30, less than about 20, or less than about 10 amino acids, such as 1 amino acid, 1-2 amino acids, 1-3 amino acids, 1-4 amino acids, 1-5 amino acids.

The term “homologue(s)” as used herein in the context of a polypeptide sequence, such as a SEQ ID NO 1 and SEQ ID NO 2 refers to a polypeptide which is at least about 70% homologous, such as at least about 80% homologous, such as at least about 85% homologous, or at least about 90% homologous, such as at least about 95%, about 96%, about 97%, about 98% or about 99% homologous to said polypeptide sequence. Homology between two polypeptide sequences may be determined using ClustalW alignment algorithm using standard settings, as referred to herein.

The term “fragment(s)” as used herein in the context of a polypeptide sequence, such as a SEQ ID NO 1 and SEQ ID NO 2 refers to a polypeptide which consists of only a part of the polypeptide sequence. A fragment may therefore comprise or consist of at least about 50%, such as at least about 60%, such as at least about 70%, such as at least about 80%, such as at least about 90% or such as at least about 95% of said polypeptide sequence.

The variant, homologue and fragment according to the invention all retain at least part of the desired (for the purpose of the present invention) enzymatic activity of the parent enzyme, such as at least about 10%, at least about 20%, at least about 30%, at least about 40%, at least about 50%, at least about 60%, at least about 70%, at least about 80% at least about 90% or all the enzyme activity of the parent enzyme.

It will be recognised by the skilled person that when we identify preferred enzymes for use in the composition and methods of the invention by their specific SEQ IDs, this includes enzymes which are derived from the nucleic acids which encode the corresponding amino acid SEQ IDs when expressed, either in their native host species or a heterologous host species, and as such the enzymes may be co- or post-translationally processed.

The term “about” as used herein to refer to concentrations, activities and conditions etc. includes and specifically discloses the exact values and exact ranges referred to.

The term “polyol” as used herein refers to a sugar alcohol which comprises more than one hydroxyl group. Polyol is a distinct term from sugar as polyols only contain hydroxyl (COH) groups and belong to the general group alditols, whilst sugars have carbonyl groups (COOH).

Disaccharides and monosaccharides can both form sugar alcohols; however, sugar alcohols derived from disaccharides (eg. Maltitol and lactitol) are not entirely hydrogenated because only one aldehyde group is available for reduction. In one embodiment the sugar alcohol are fully hydrogenated.

Sugar alcohols are commonly added to foods because of their lower caloric content than sugars; however they are also generally less sweet, and are often combined with high intensity sweeteners. They are also added to chewing gum because they are not metabolized (ie broken down) by bacteria in the mouth, so they do not contribute to tooth decay. Maltitol, sorbitol and Isomalt are some of the more common types. Sugar alcohols may be formed under mild reducing conditions from their analogue sugars.

As used herein, the term “oxidase” refers to enzymes that catalyze an oxidation/reduction reaction involving molecular oxygen (O₂) as the electron acceptor. In these reactions, oxygen is reduced to water (H₂O) or hydrogen peroxide (H₂O₂). The oxidases are a subclass of the oxidoreductases.

The term “polyol oxidase” refers to an enzyme which is capable of oxidizing a polyol to the corresponding sugar. The oxidation of the polyol by the action of the polyol oxidase results in the production of hydrogen peroxide.

As used herein, the term “glucose oxidase” (“Gox”) refers to the oxidase enzyme (EC 1.1.3.4) that binds to beta-D-glucose (i.e., an isomer of the six carbon sugar, glucose) and aids in breaking the sugar down into its metabolites. GOx is a dimeric protein which catalyzes the oxidation of beta-D-glucose into D-glucono-1,5-lactone, which then hydrolyzes to gluconic acid with concomitant reduction of molecular oxygen to hydrogen peroxide.

As used herein, the term “alcohol oxidase” (“Aox”) refers to the oxidase enzyme (EC 1.1.3.13) that converts an alcohol to an aldehyde with concomitant reduction of molecular oxygen to hydrogen peroxide.

As used herein, the term “choline oxidase” (“Cox”) refers to an oxidase enzyme (EC 1.1.3.17) that catalyzes the four-electron oxidation of choline to glycine betaine, with betaine aldehyde as an intermediate with concomitant reduction of two molecules of molecular oxygen to two molecules of hydrogen peroxide.

As used herein, the term “hexose oxidase” (“Hox”) refers to an oxidase enzyme (EC 1.1.3.5) the oxidation of mono- and disaccharides to their corresponding lactones, with concomitant reduction of molecular oxygen to hydrogen peroxide. Hexose oxidase is able to oxidize a variety of substrates including D-glucose, D-galactose, maltose, cellobiose, and lactose, etc.

As used herein, “glycerol oxidase” refers to an oxidase enzyme (EC 1.1.3.) that catalyzes the oxidation of glycerol to glyceraldehyde, with concomitant reduction of molecular oxygen to hydrogen peroxide.

It is recognised that the activity of an enzyme will depend on the conditions and substrates available, and therefore the activity of an enzyme may differ from a standard assay condition (in vitro assay), as compared to within a composition according the invention, or the use of such a composition in the desired application. In one embodiment, the enzyme activity is determined by an in vitro assay as referred to in the examples. Alternatively, the activity of an enzyme is determined in situ, i.e. in the composition according to the invention and under conditions which the composition is to be used. The same analytic methods may be used for determining the in situ enzyme activity as for the in vitro activity, just the assay is performed using the composition matrix, or under conditions in which the composition/product according to the invention are used.

The term “sugar” as used herein refers to monosaccharides, disaccharides and oligosaccharides, hexose, including but not limited to sugars selected from the group consisting of lactose, maltose, sorbose, triose, pentose, hexose, mannose, glucose, galactose, xylose, fructose, isomaltose, erythrose.

Sugars contain either aldehyde groups (—CHO) or ketone groups (C═O), where there are carbon-oxygen double bonds, making the sugars reactive. Preferably, the term sugar conforms to (CH2O)n where n is between 3 and 6, such as 3, 5 or 6, or 5 or 6.

The sugars may be trioses, pentose or hexose, preferably pentose or hexose sugars, preferably in closed-chain form.

The sugar may be selected from the group consisting of: sucrose, fructose, glucose, galactose, maltose, lactose and mannose.

By “oral care product” as used herein is meant a product which is not intentionally swallowed for purposes of systemic administration of therapeutic agents, but is retained in the oral cavity for a sufficient time to contact substantially all of the dental surfaces and/or oral mucosal tissues for purposes of oral activity. In the context of the present invention oral care product also includes products for cleaning dentures, artificial teeth and the like. The oral care product may have any suitable physical form including but not limited to e.g. powder, paste, gel, liquid, ointment, chewing gum tablet or spray.

As used herein, “cleaning compositions” and “cleaning formulations” refer to compositions that find use in the removal of undesired compounds from items to be cleaned, including but not limited to fabric, dishes, contact lenses, other solid substrates, hair (shampoos), skin (soaps and creams), teeth (mouthwashes, toothpastes) etc. The term encompasses any materials/compounds selected for the particular type of cleaning composition desired and the form of the product (e.g., liquid, gel, granule, or spray composition), as long as the composition is compatible with the oxidase and other enzyme(s) used in the composition, and any reversible enzyme inhibitors in the composition. The specific selection of cleaning composition materials are readily made by considering the surface, item or fabric to be cleaned, and the desired form of the composition for the cleaning conditions during use.

The terms further refer to any composition that is suited for cleaning, bleaching, disinfecting, and/or sterilizing any object and/or surface. It is intended that the terms include, but are not limited to detergent compositions (e.g., liquid and/or solid laundry detergents and fine fabric detergents; hard surface cleaning formulations, including but not limited to for glass, wood, ceramic and metal counter tops and windows; carpet cleaners; oven cleaners; fabric fresheners; fabric softeners; and textile and laundry pre-spotters, as well as dish detergents).

Indeed, the term “cleaning composition” as used herein, includes unless otherwise indicated, granular or powder-form all-purpose or heavy-duty washing agents, especially cleaning detergents; liquid, gel or paste-form all-purpose washing agents, especially the so-called heavy-duty liquid (HDL) types; liquid fine-fabric detergents; hand dishwashing agents or light duty dishwashing agents, especially those of the high-foaming type; machine dishwashing agents, including the various tablet, granular, liquid and rinse-aid types for household and institutional use; liquid cleaning and disinfecting agents, including antibacterial hand-wash types, cleaning bars, mouthwashes, denture cleaners, car or carpet shampoos, bathroom cleaners; hair shampoos and hair-rinses; shower gels and foam baths and metal cleaners; as well as cleaning auxiliaries including but not limited to bleach additives and “stain-stick” or pre-treat types.

As used herein, the terms “detergent composition” and “detergent formulation” are used in reference to mixtures which are intended for use in a wash medium for the cleaning of soiled objects. In some preferred embodiments, the term is used in reference to laundering fabrics and/or garments (e.g., “laundry detergents”). In alternative embodiments, the term refers to other detergents, including but not limited to those used to clean dishes, cutlery, etc. (e.g., “dishwashing detergents”). It is not intended that the present invention be limited to any particular detergent formulation or composition. Indeed, it is intended that in addition to oxidase, the term encompasses detergents that contain surfactants, transferase(s), hydrolytic enzymes, oxido reductases, perhydrolases builders, bleaching agents, bleach activators, bluing agents and fluorescent dyes, caking inhibitors, masking agents, enzyme activators, enzyme inhibitors, antioxidants, and solubilizers. In some preferred embodiments, the detergent formulations include, but are not limited to those set forth in U.S. patent application Ser. Nos. 10/576,331 and 10/581,014, as well as WO 05/52161 and WO 05/056782 find use in the present invention. However, it is not intended that the present invention be limited to any particular detergent formulation(s), as any suitable detergent formulation finds use in the present invention.

As used herein, “dishwashing composition” refers to all forms of compositions for cleaning dishware, including cutlery, including but not limited to granular and liquid forms. It is not intended that the present invention be limited to any particular type or dishware composition. Indeed, the present invention finds use in cleaning dishware (e.g., dishes, including, but not limited to plates, cups, glasses, bowls, etc.) and cutlery (e.g., utensils, including but not limited to spoons, knives, forks, serving utensils, etc.) of any material, including but not limited to ceramics, plastics, metals, china, glass, acrylics, etc. The term “dishware” is used herein in reference to both dishes and cutlery.

As used herein, “wash performance” of an enzyme refers to the contribution of an enzyme to washing that provides additional cleaning performance to the detergent without the addition of the enzyme to the composition. Wash performance is compared under relevant washing conditions.

The term “relevant washing conditions” is used herein to indicate the conditions, particularly washing temperature, time, washing mechanics, sud concentration, type of detergent and water hardness, actually used in households in a detergent market segment.

The term “improved wash performance” is used to indicate that a better end result is obtained in stain removal from items washed (e.g., fabrics or dishware and/or cutlery) under relevant washing conditions, or that less enzyme, on weight basis, is needed to obtain the same end result relative to another enzyme.

The term “retained wash performance” is used to indicate that the wash performance of an enzyme, on weight basis, is at least 80% relative to another enzyme under relevant washing conditions.

Wash performance of enzymes is conveniently measured by their ability to remove certain representative stains under appropriate test conditions. In these test systems, other relevant factors, including but not limited to detergent composition, sud concentration, water hardness, washing mechanics, time, pH, and/or temperature, can be controlled in such a way that conditions typical for household application in a certain market segment are imitated.

As used herein, the term “disinfecting” refers to the removal of contaminants from the surfaces, as well as the inhibition or killing of microbes on the surfaces of items. It is not intended that the present invention be limited to any particular surface, item, or contaminant(s) or microbes to be removed.

### Control of the Reaction

Both oxidation reactions require a presence of oxygen and would thus not take place to a significant degree as long as the composition is contained in a sealed container, including but not limited to in a controlled oxygen environment (oxygen limited environment), including but not limited to in the exclusion of limitation or absence of molecular oxygen. Once the container is open and the composition is subjected to atmospheric air or alternative source of molecular oxygen, the reaction can take place.

The invention therefore also provides for a packaged product comprising the composition of the invention, wherein the composition is maintained in a controlled oxygen environment, or a oxygen limited environment, including but not limited to in the absence of (available) molecular oxygen, so as to prevent or reduce the production of hydrogen peroxide within said packaged product.

An alternative method is providing the composition of the invention in a controlled water environment, where the level of water is sufficiently low to prevent or reduce the production of hydrogen peroxide within the composition or a packaged product. Suitably the level of water in such a composition may be less than about 2%, such as less than about 1%, such as less than about 0.5%, such as less than about 0.2% or less than about 0.1%.

Alternatively the reaction may be prevented from occurring prematurely by physically separating the enzyme and substrate components from each other by compartmentalisation methods well known in the art. In one embodiment, the reaction is prevented by separating the first substrate from the composition compartment comprising the first oxidase and the further oxidoreductase. The first substrate may be added to activate the composition, or may form part of the application matrix, either as a routine ingredient of said application matrix, or supplemented to said application matrix either before, during or prior to the addition of the composition. The invention therefore also provides for a first composition comprising the first oxidase (such as polyol oxidase) and the further oxidoreductase, as referred to herein, in a kit of parts, with a further composition comprising said first substrate, wherein said first and second compositions are separated from one another.

Although it is envisaged that the composition of the invention may be provided as a two or more pot system, for combination upon use, it is also considered that technologies such as encapsulation or micro-encapsulation may be employed to keep the enzyme component of the composition separated from the substrates. The release from the micro-encapsules may be triggered by, for example mechanical force or dilution upon use of the composition of the invention.

In one aspect the composition according to the invention comprises a kit of parts comprising at least two pots, wherein the first pot comprises the first oxidase and the further oxidoreductase, and a second pot which comprises the first substrate.

In the applications disclosed herein, including but not limited to in cosmetic applications (also in oral care application), in one embodiment it is advantageous to compartmentalise the composition of the invention, so that the production of hydrogen peroxide is achieved upon application, for example by mechanical scrubbing allowing the release of the enzyme system and letting it come into contact with first substrate in situ during application, but not during storage.

In one aspect of the invention the pH of the composition is between 5 and 9, preferably between 6 and 8. When the composition according to the invention is an oral care product it is preferred to use a first oxidase, such as a polyol oxidase, such as SOX and an oxidoreductase, which is substantially active at the pH prevailing in the mouth i.e. at a pH between (about) 5 and (about)₉, preferably between about 6 and about 8. It is furthermore preferred that the first oxidase, such as polyol oxidase, such as sorbitol oxidase and the oxidoreductase are active at ambient temperature, or at body temperature.

In one embodiment the first oxidase, and or further oxidoreductase, and any other enzymes present may be immobilized.

### Further Enzymes and Enzyme Systems

The composition according to the invention may comprise further enzymes including but not limited to one or more enzymes selected from the group consisting of: peroxidases including but not limited to lactoperoxidase, laccases, amyloglucosidase, amylases including but not limited to maltogenic and non-maltogenic amylases, including but not limited to NovaMyl™, glucoamylase, dextrinase, protease, lysozyme, mutanase, lipase, acyl-transferase, including but not limited to the acyl-transferases disclosed in WO2004064537, and xylanase.

In one embodiment, the composition of the invention, including but not limited to the oral composition of the invention, or alternative compostions as referred to herein, comprise thiocyanate, thereby allowing the conversion of thiocyanate to hypothiocyanate due to the production of hydrogen peroxide. For oral care products, there is, typically, sufficient lactoperoxidase present in the saliva in the mouth to convert thiocyanate to hypothiocyanate in the presence of hydrogen peroxide. However, the composition according to the invention may also comprise lactoperoxidase, to facilitate this conversion, thereby allowing the production of an effective antibacterial agent in situ.

It is recognized that the rate of the release of hydrogen peroxide may become limited if the availability of oxygen is limiting. It is therefore considered that coupling of the present enzyme system to a oxygen generation system may be appropriate.

### Applications

The composition according to the invention can be used to treat any type of material where whitening and/or bleaching is desired including but not limited to e.g. teeth, paper and textiles.

An advantage of the use of this combination of enzymes for bleaching and/or whitening is that the whitening and/or bleaching effect is obtained by the use of non-hazardous materials compared to previous use of bleaching agents in e.g. oral care products.

The composition according to the invention has furthermore an anti-microbial effect due to the resulting hydrogen peroxide. A further aspect of the invention relates to the use of the composition according to the invention in the manufacture of an oral care product for the treatment or prevention of microbial effects relating to breath malodour or periodontitis.

Other beneficial uses of the present composition are in food preservation where the composition acts as an oxygen scavenger using two moles of O₂ every time one mole of sugar is used.

In the personal care area, the composition of the present invention may be added to toothpaste, in particular, whitening teeth, mouthwash, denture cleaner, liquid soap, skin care creams and lotions, hair care and body care formulations and solutions for cleaning contact lenses in an amount effective to act as an antibacterial agent. The composition of the present invention may also be a component of a laundry detergent composition or a dishwashing detergent composition and may be used as a hydrogen peroxide source. The laundry detergent composition may comprise a surfactant, said the composition of the present invention. The dishwashing detergent composition may comprise said the composition of the present invention and a bleach precursor or peroxy acid. The composition of the present invention may particularly be useful for removing stains.

### Detergents

The detergent (product) according to the invention comprises the composition according to the invention. Therefore, in one embodiment the invention provides for the use of the composition of the invention in a detergent product, including but not limited to a cleaning composition, cleaning formulation, detergent composition or detergent formulation.

The detergent (product) may be used as a bleaching or whitening agent, or may be used as a disinfecting agent, or both bleaching/whitening and disinfecting agent.

The detergent (product) may comprise further enzymes or enzyme systems, as disclosed herein in, in particular peroxidases, proteases, amylases, lipases, acyl-transferases and lactoperoxidases (optionally in the presence of thiocyanate).

In one embodiment, the detergent product may be in the form of a cleaning composition or cleaning formulation.

In one embodiment, the detergent product may be in the form of a detergent composition or detergent formulation.

The detergent product may be a dishwashing composition.

Preferably the detergent product according to the invention has an improved wash performance, under the relevant washing conditions, as compared to an equivalent product which does not comprise the composition of the invention.

In some embodiments, detergent formulations comprising the composition of the invention, and a bleach booster are used to produce active oxygen species in laundry wash liquor to bleach stains.

In some embodiments, the detergent composition further comprises an acyl-transferase and its substrate are used to produce active oxygen species in laundry wash liquor to bleach stains.

In a one aspect the composition is an edible composition, including but not limited to an oral care composition, an (orally administered) medicament composition, or a food or feed composition.

The term ‘edible composition’ refers to compositions which are for oral administration or for consumption via the oral cavity.

The edible compositions may comprise one or more of flavourings, preservatives, textural ingredients, emulsifiers, sweeteners, humectants, and/or binding agents which (typically) have been approved for human (or animal) consumption.

### Oral Care

In one aspect of the invention an oral care product comprising a composition according to the invention and ingredients used in oral care products, is provided.

In a further aspect of the invention the oral care product according to the invention comprises a first enzyme, such as polyol oxidase, such as sorbitol oxidase, a further enzyme, such as further oxidoreductase and a polyol, such as sorbitol and ingredients used in oral care products where antimicrobial effect and/or whitening and/or bleaching is desired, is provided.

Examples of oral care products include toothpaste, dental cream, gel or tooth powder, odontic, mouth rinses, mouth sprays, pre- or post brushing rinse formulations, chewing gum, lozenges, and candy.

The oral care product may comprise further active ingredients including but not limited to thiocyanate, zinc gluconate, lysozyme, lactoferrin, lactoperoxidase, and amyloglucosidase. Further ingredients are listed in WO97/06775, and include redox mediators.

In one embodiment the oral care composition/product according to the invention further comprises fluoride.

Toothpastes and tooth gels typically include ingredients including but not limited to abrasive polishing materials, foaming agents, flavouring agents, humectants, binders, thickeners, sweetening agents, whitening/bleaching/stain removing agents, water, and optionally enzymes.

Mouth washes, including plaque removing liquids, typically comprise ingredients including but not limited to a water/alcohol solution, flavour, humectant, sweetener, foaming agent, colorant, and optionally enzymes.

Chewing gum: Suitable compositions for the preparation of a chewing gum are disclosed in WO2005/006872 and U.S. Pat. No. 4,564,519.

When used in oral care products the amount of first oxidase, such as polyol oxidase, including but not limited to sorbitol oxidase, and further enzyme (such as further oxidoreductase) should be a safe and effective amount which means an amount high enough to significantly modify the condition to be treated or effect the desired whitening and/or bleaching result but low enough to avoid serious side effects.

In a further aspect the invention provides a method for bleaching and/or whitening of teeth, comprising contacting the teeth with an oral care product comprising a composition according to the invention in an amount and time suitable for bleaching and/or whitening teeth.

The present invention provides a safe teeth whitening composition that has the advantage over the prior art by providing use of a first oxidase, such as a polyol oxidase, such as sorbitol oxidase which can act on substrates which are non-cariogenic (i.e. substrates which do not degrade into cariogenic sugars including but not limited to sucrose, glucose, fructose, maltose etc.).

### Food and Feed

The composition of the invention may be used in general disinfecting of food and feed and food and feed environments, including but not limited to manufacturing, processing an preparation facilities, including but not limited to milking parlours, cheese making facilities, meat processing factories, vegetable and fruit washing and processing plants, in restaurants etc.

In meat (i.e. animal meat, including fish) processing factories, the composition of the invention may be used in washing and disinfecting animal carcasses and food products derived therefrom. The use of the polyol oxidase/further enzyme system is particularly preferred as this reduces the level of fermentable sugars present in or on the surface of the meat products, reducing the growth of undesirable micro-organisms. The composition according to the invention may further comprise alkali silicates (US2004062851, hereby incorporated by reference), such compositions are considered particularly useful in decontaminating meat products. The use of alkali silicates also increased the water retention properties of meant and meat products, and can be used to enhance the retention of the composition of the invention in meat and meat products.

A further advantage is the lowering of the pH due to the generation of acidic further products, which also inhibits the generation of micro-organisms in or on the surface of the meat. Similar benefits are seen in vegetable and fruit washing and processing facilities.

The composition of the invention may also be used in food and feed products. Hydrogen peroxide is used extensively as an anti-microbial agent in the production of dairy products and egg products. Suitably, the composition according to the invention may be added to, or may be a food product selected from the group consisting of: dairy products, including but not limited to milk, cream, cheese, whey, yoghurt, butter, or egg, including but not limited to egg yolk or egg white.

The lowering of the pH due to the accumulation of acidic further products is also a considerable advantage in other food types, including but not limited to in cheese production, where the rapid lowering of the pH increases the rate of maturation of cheese, including but not limited to cheddar cheeses and Italian hard cheeses, reducing the maturation and storage time. The lower pH therefore also contributes to an enhanced taste of cheese.

In one embodiment the composition of the invention is used in a beverage, including but not limited to a fruit juice. Fruit juices typically comprise suitable first substrates, therefore it may be unnecessary to add further first substrate to the fruit juice or alternatively they may be supplemented with the first substrates referred to herein.

Furthermore. O₂ is scavenged by the production of xylose, which can prevent food spoilage. A further benefit is the lowering of pH compared to a system where SOX is used alone, where no such lowering of pH is observed—the lowering of pH can also prevent or reduce food spoilage.

### Personal Care Products

In the personal care area, the composition of the present invention may be added to a personal care product other than oral care products, including but not limited to denture cleaner, liquid soap, skin care creams and lotions, hair care and body care formulations and solutions for cleaning contact lenses in an amount effective to act as an antibacterial agent.

### Paper Production

The composition of the invention may also be used for cleaning paper mills, the hydrogen peroxide produced oxidizes carbohydrates to the corresponding acids, which in turn are believed to chelate with the cationic part of inorganic salts such as scale. This enables better cleaning and/or control of sediments, and for water streams it leads to a turbidity reduction, to an improved settling behaviour, as well as to a colour reduction, all of which makes cleaning and effluent control procedures easier and less expensive (see WO06/061018).

### Cosmetics

Glucose oxidase has been used as the basis of a commercial preservative system for cosmetics and toiletries. However, the present invention provides for a suitable replacement for the use of glucose oxidase as it provides a far more effective system for producing hydrogen peroxide, and/or may also provide an effective anti-microbial/anti-bacterial action when in use. It may also provide a dual function product, being a cosmetic which also bleaches or whitens the skin during use.

The lactoperoxidase system is used in numerous cosmetic products. The cosmetic composition or product according to the invention may therefore further comprise lactoperoxidase and thiocyanate.

Polyols, such as sorbitol is typically a major component of cosmetics, or may be added to cosmetics to provide a suitable substrate for the polyol oxidase in the composition according to the invention.

The use of the composition of the invention in cosmetic products may provide one or more of the following benefits: preservative of the cosmetic, and microbial/bacterial activity when applied to the skin, skin lightening/bleaching effect.

Cosmetic ingredients include: Antioxidants, binding agents, emollients, emulsifiers, humectants, pigments, lubricants, preservatives, solvents, fragrances, surfactants, vehicle substances, including but not limited to an inert base material, stabilizers and thickeners.

The composition/product of the invention may be used in the form of one or more of the cosmetic products selected from the group consisting of: Lipstick, lip gloss, lip pencil, and Lip-Ink; liquid foundation; Cream foundation; Powder; Rouge (blush or blusher); bronzer; Mascara; Eyeliner and eye shadow; Nail polish; Concealer; skin care products including but not limited to creams and lotions, e.g. to moisturize the face and body; sunscreens and sun lotions; treatment products to repair or hide skin imperfections;

Cosmetics can also be described by the form of the product, as well as the area for application. Cosmetics can be liquid or cream emulsions; powders, both pressed and loose; dispersions; and anhydrous creams or sticks.

The composition/products according to the invention may also be used in cosmetic techniques including but not limited to skin beaching and/or whitening.

### Skin Beaching

Hydroquinone has been used as an ingredient in preparations for skin beaching. However, recently the US Environmental Protection Agency has issued a notice of proposed rulemaking that would establish that over-the-counter (OTC) skin bleaching drug products based on hydroquinone are potentially carcinogenic and not generally recognized as safe and effective (GRAS) and are misbranded.

The compositions and products according to the invention may be used for the preparation of enzyme based products for skin beaching, and therefore provide a safe alternative to the use of hydroquinone.

Skin bleaching products may comprise other bleaching agents, including but not limited to licorice extract, mulberry extract, arbutin, kojic acid, bearberry extract, AHA blends, salicylic acids, aloeleic acid, citric acids, lactic acids, as well as suitable base matrixes, and sunscreens.

### Hair Bleaching

For hair colouring bleaching a chemical oxidizing agent is used. Suitable oxidizing agents are persulfates, chlorites and, above all, hydrogen peroxide or addition products thereof with urea, melamine and sodium borate. Therefore the composition of the present invention may also be used as a safe alternative source of hydrogen peroxide for hair bleach.

Suitably a hair beaching composition may also be used for colouring keratin fibers, and may therefore contain at least one dye precursor as well as the composition of the present invention. The present invention also relates to a process for coloring keratin containing fibers using the composition of the present invention.

### Paint

The term ‘paint’ as used in the context of the present invention herein refers to the family of products used to protect and/or add color to an object or surface by covering it with a pigmented or non-pigmented coating, and includes varnish, wood stains, shellac, lacquer, and enamel. Paint can be applied to almost any kind of object. Paint is a semifinished product, as the final product is the painted article itself.

The paint may be for used in application where the dried paint is exposed to a natural water environment, including but not limited to a maritime paint. Maritime paints are used to prevent or reduce anti-fouling on the hulls of boats and ships caused by the growth of organisms including but not limited to algar, barnacles and the like.

The paint may be a decorative paint, for example used in the interior or exterior of buildings and other objects.

The paint may be a protective paint, preventing or reducing microbial spoilage of the surface or material upon which the paint is applied, including but not limited to wet rot or dry rot.

The composition according to the invention can be used as a preservative in paint. Alternatively or in addition, the composition can be used in a paint for reducing fouling, including but not limited to in maritime paint, where the production of hydrogen peroxide causes an oxidative layer that prevent or reduces the attachment or growth of fouling organisms on the surface. Such paints which comprise the composition according to the invention offer a beneficial enzyme solution to this problem as the hydrogen peroxide is produced without the addition, or significant accumulation of fermentable substrates within the paint, which can actually encourage anti-fouling, especially if the enzymes within the paint become inactivated.

### Pesticides

Pesticide products usually contain no more than 35% hydrogen peroxide, which is then usually diluted to 1% or less when applied as a spray or a liquid. Hydrogen peroxide is used for many non-food and food crops (e.g., fruits, nuts, and vegetables), both indoors and outdoors, and before and after harvest and for disinfecting food storage facilities. The target Pests are typically microbes, including fungi and bacteria which cause plant diseases. The hydrogen peroxide containing pesticides, used to prevent and control plant pathogens, are typically applied as a spray on foliage, or as a dip on cuttings and roots, or as a pre-planting soil treatment.

The composition according to the invention may therefore be used as a pesticide, either directly, allowing production of hydrogen peroxide in situ, or as a system for generation of hydrogen peroxide which may subsequently be applied to the appropriate surface.

### FURTHER EMBODIMENTS

1. A composition comprising

a sorbitol oxidase, a first substrate, and an oxidoreductase,
wherein the first substrate is oxidisable by the sorbitol oxidase to form hydrogen peroxide and a second substrate, and the second substrate is oxidisable by the oxidoreductase to form hydrogen peroxide and a product.

2. The composition according to the invention such as to embodiment 1, wherein the first substrate is one or more selected from the group consisting of D-sorbitol, D-xylitol, D-mannitol, D-arabitol, glycerol, inositol, 1,3-propanediol, 1,3-butanediol, and 1,4-butanediol.

3. The composition according to the invention such as to embodiment 2, wherein the first substrate is one or more selected from the group consisting of D-sorbitol or D-xylitol.

4. The composition according to the invention such as to embodiment 3, wherein the first substrate is D-sorbitol.

5. The composition according to the invention such as to embodiment 1, wherein the oxidoreductase is one or more selected from the group consisting of hexose oxidase, glucose oxidase, carbohydrate oxidase, and oligosaccharide oxidase.

6. The composition according to the invention such as to embodiment 5, wherein the oxidoreductase is one or more selected from the group consisting of a carbohydrate oxidase, a hexose oxidase or a glucose oxidase.

7. The composition according to the invention such as to embodiment 6, wherein the oxidoreductase is a hexose oxidase.

8. The composition according to the invention such as to embodiment 1, wherein the sorbitol oxidase is derived from a strain of Streptomyces.

9. The composition according to the invention such as to any one of the embodiments 1-8,

wherein the pH of the composition is between 5 and 9.

10. The composition according to the invention such as to any one of the embodiments 1-9, wherein the pH of the composition is between 6 and 8.

11. The composition according to the invention such as to any one of the embodiments 1-10, wherein the sorbitol oxidase and the oxidoreductase are active at ambient temperature.

12. An oral care product comprising a composition according to the invention such as to any one of embodiments 1-11 and ingredients used in oral care products.

13. The use of a sorbitol oxidase for whitening and/or bleaching.

14. The use of a composition according to the invention such as to any one of the embodiments 1-10 for whitening and/or bleaching.

15. The use according to the invention such as to any one of the embodiment 13-14 for whitening and/or bleaching teeth.

16. A method for bleaching and/or whitening of teeth, comprising contacting the teeth with an oral care product comprising a composition according to the invention such as to any one of the embodiments 1-10 in an amount and time suitable for bleaching and/or whitening teeth.

17. Sorbitol oxidase and D-xylitol for use as a medicament.

18. A medicament according to the invention such as to embodiment 17, wherein the sorbitol oxidase is derived from a strain of Streptomyces.

19. An oral care product comprising sorbitol oxidase and D-xylitol, and ingredients used in oral care products.

20. The use of a sorbitol oxidase and D-xylitol for whitening and/or bleaching of teeth.

Having thus described in detail preferred embodiments of the present invention, it is to be understood that the invention defined by the above paragraphs is not to be limited to particular details set forth in the above description as many apparent variations thereof are possible without departing from the spirit or scope of the present invention.

### Examples

The following examples are provided in order to demonstrate and further illustrate certain preferred embodiments and aspects of the present invention and are not to be construed as limiting the scope thereof.

In the experimental disclosure which follows, the following abbreviations apply: ° C. (degrees Centigrade); rpm (revolutions per minute); H₂O (water); HCl (hydrochloric acid); aa (amino acid); bp (base pair); kb (kilobase pair); kD (kilodaltons); gm (grams); μg and ug (micrograms); mg (milligrams); ng (nanograms); μl and ul (microliters); ml (milliliters); mm (millimeters); nm (nanometers); μm and um (micrometer); M (molar); mM (millimolar); μM and uM (micromolar); U (units); V (volts); MW (molecular weight); sec (seconds); min(s) (minute/minutes); hr(s) (hour/hours); MgCl₂ (magnesium chloride); NaCl (sodium chloride); OD₂₈₀ (optical density at 280 nm); OD₆₀₀ (optical density at 600 nm); EFT (“effective fermentation time”); HDL (Heavy Duty Detergent Liquid); EtOH (ethanol); PBS (phosphate buffered saline [150 mM NaCl, 10 mM sodium phosphate buffer, pH 7.2]); SDS (sodium dodecyl sulfate); Tris (tris(hydroxymethyl)aminomethane); TAED (N,N,N′N′-tetraacetylethylenediamine); w/v (weight to volume); v/v (volume to volume); GOX and GOx (glucose oxidase); AOX and AOx (alcohol oxidase); COX and Cox (choline oxidase); HOX and HOx (hexose oxidase); SOX and Sox (sorbitol oxidase); AATCC (American Association of Textile and Coloring Chemists); WFK (wfk Testgewebe GmbH, Bruggen-Bracht, Germany); Testfabrics (Testfabrics Inc, Pittston Pa.); ATCC (American Type Culture Collection, Manassas, Va.); Geneart (Geneart, Regesburg, Germany); Invitrogen (Invitrogen, Inc., Carlsbad, Calif.); Baker (J. T. Baker, Phillipsburg, N.J.); NAEF (NAEF, Press and Dies, Inc., Bolton Landing, N.Y.); Fluka (Fluka Chemie AG, Buchs, Switzerland); Prometric (Prometic Biosciences, Wayne N.J.); Minolta (Konica Minolta. Glen Cove, N.Y.); and Sigma (Sigma-Aldrich Chemical Co., St. Louis, Mo.).

### Example 1

### Construction of Strains Expressing Sorbitol Oxidase of Streptomyces sp. h-7775 in Streptomyces lividans

The protein sequence (SEQ ID NO: 1) of the sorbitol oxidase was obtained from the published amino acid sequence (See e.g., Hiraga et al., Biosci. Biotechnol. Biochem., 62: 4347-353 [1998]). The signal sequence of the twin-arginine pathway of the Streptomyces ceolicolor SCO6772 gene (SEQ ID NO:3) was obtained from complete genome sequence of Streptomyces coelicolor.

The sorbitol oxidase was expressed in Streptomyces as a fusion protein of the signal sequence of the SCO6772 protein (SEQ ID NO:3) and sorbitol oxidase (SEQ ID NO:1). A restriction site for NcoI was introduced at the 5′ end of DNA for cloning purposes, which resulted addition of an amino acid glysine residues at position 2 (See, SEQ ID NO:4).

A restriction site for BamHI was also introduced at the 3′ end of DNA for cloning purposes. The codons of the fusion gene were optimized for expression in Streptomyces lividans. DNA was synthesis by Geneart. The DNA fragment spanning the two restriction sites (i.e., from NcoI to BamHI (SEQ ID NO:5)) was cloned into Streptomyces expression plasmid pKB105 (See, U.S. patent application Ser. No. 11/303,650, filed Dec. 16, 2005, incorporated by reference in its entirety) which was cut with BamHI completely and NcoI partially.

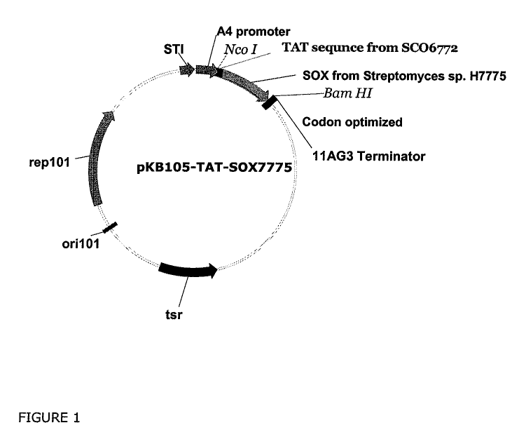

The expression plasmid (pKB105-TAT-SOX-7775; FIG. 1) was transformed into Streptomyces lividans strain g3s3 (See, U.S. patent application Ser. No. 11/305,650, supra) and three transformants were selected and grown in TS medium for 2-3 days in the presence of 50 ug/ml thiostrepton at 30° C. Cells were then transferred to a production medium free of antimicrobials and growth was continued for another three days. Then, 1 ml of the culture was transferred to each of two culture tubes and the cells were removed by centrifuge under conditions sufficient to separate the cells from the supernatants. The supernatants obtained from these two culture tubes were tested in enzyme activity assays. Two oligos (SEQ ID NO:6 and SEQ ID NO:7) were obtained from Invitrogen.

| | |
|---|---|
| GCGCTAGCCGGCCCCCCGGCACAGGCCATGACCCCGGCCGAGAAGAACTGGG | (SEQ ID NO:6) |
| CAGGAAACAGCTATGAC | (SEQ ID NO:7) |

The primers were used in PCR to amplify sorbitol oxidase gene and to fuse the sorbitol oxidase gene to the celA signal sequence. The PCR reaction mixture containing DNA, dNTPs, primer and 4% DMSO in 1× buffer was heated to 98° C. for 4 minutes to denature the DNA templates. Herculase® II enzyme (Stratagene) was added to the tube and PCR reaction was performed in 30 cycles of 98° C. for 30 seconds, 62° C. for 30 seconds and 72° C. for 1 minute and 8 seconds. The final extension at 72° C. was done for 5 minutes and the reaction was chilled to 4° C.

The resulting PCR fragment contained a portion of the celA signal sequence, the sorbitol oxidase gene, and a portion of vector sequence containing two restriction enzyme sites (SEQ ID NO:8).

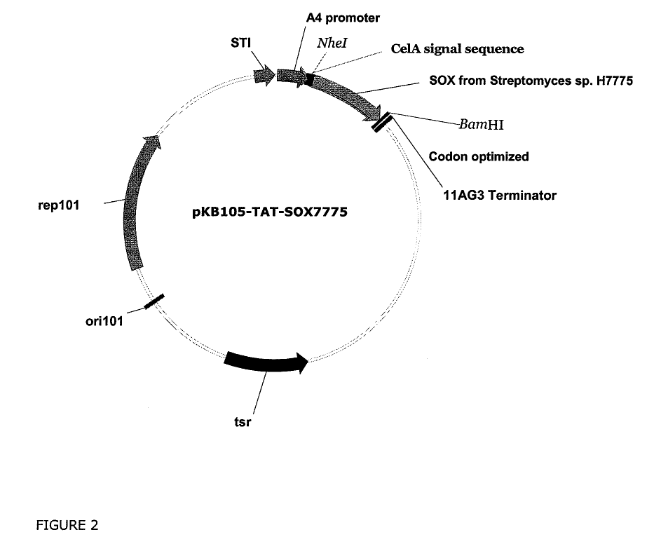

The PCR fragment was digested with restriction enzymes NheI and BamHI to remove the vector sequence portion. The resulting fragment was then cloned to expression vector pKB105 to generate the plasmid “pKB105-CelA-Sox7775” (See, FIG. 2). The expression plasmid was transformed into Streptomyces lividans strain g3s3 and three transformants were selected and grown in TS medium for 2-3 days in the presence of 50 ug/ml thiostrepton at 30° C. Cells were then transferred to a production medium free of antibiotics and growth was continued for another three days. Then, 1 ml sample was transferred to each of two new culture tubes and cells were removed and the enzyme was purified as described in Example 2.

### Example 2

### Expression of the Sorbitol Oxidase Gene from Streptomyces sp. H-7775 in E. coli Strain BL21(DE3)pLysS

The amino acid sequence of the sorbitol oxidase gene from Streptomyces sp. H-7775 SOX gene, published by Hiraga et al. 1998. Bioscience Biotech Biochem. 61:1699-1704, 1998 was retrieved from the sequence database.

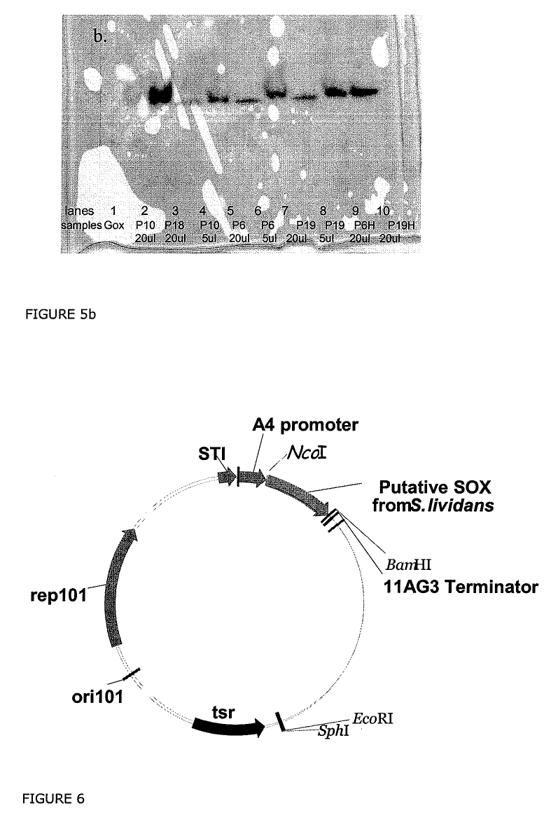

A synthetic gene (with neutral codons) encoding the H-7775 SOX gene was used to express the sorbitol oxidase gene in E. coli strain BL21(DE3)pLysS. The expression vector pET 24a with the SOX gene was cloned as NdeI+Bam H1 fragment. The resulting plasmid (FIG. 5a) was transformed and propagated in E. coli Top10 cells (Invitrogen, USA). Kanamycin resistant transformants containing the 1.2 kb SOX gene were identified by the direct colony PCR method. The SOX positive transformants were cultivated and plasmid DNA isolated. Plasmid DNA containing the cloned SOX gene was then used to transform the host strain BL21(DE3)pLysS. The entire transformation reaction was directly used to inoculate a 250 ml flasks containing 25 ml LB+antibiotics kanamycin (50 ug/ml) and chloramphenicol (34 ug/ml). The cultures were incubated overnight with shaking at 37° C. A 2 liter flasks containing 500 mls of LB containing kanamycin (50 ug/ml) and chloramphenicol (34 ug/ml) was inoculated with 25 mls of the overnight culture and was further allowed to grow for 2 hours to reach approximately OD₆₀₀ 0.4-0.6 mid logarithmic phase. IPTG to a final concentration of 1 mM was added to the cultures. The cultures were further incubated for another 2 hours and then harvested by centrifugation. The resulting pellets were resuspended in phosphate buffer and the cells were passed through a French press for cell disruption/lysis. The different cell lysates were analyzed for sox activity (see Table 1 below) and fractionated by gel electrophoresis (native & denaturing conditions). The cell lysates were fractionated on a native gel that was further tested in an in-gel overlay activity assay using sorbitol as the substrate and an assay based on the PMS mediated reduction of NBT. FIG. 5b shows the presence of a discrete stained protein band corresponding to a flavoprotein, the SOX protein that has activity towards sorbitol. The PMS/NBT is a diagnostic assay for flavoproteins where PMS=phenazine methosulfate is a redox mediator which is reduced by reduced FAD. Glucose oxidase is also a flavoprotein in lane 1 FIG. 5b but is not active towards sorbitol thus the absence of stained band. P10 is transformant with the empty vector pET24a (with out SOX gene) showing the absence of active SOX protein band. The absence of a stained band in lane 1 showed that glucose oxidase is not active towards sorbitol, the substrate used in the overlay assay mix. This assay can therefore be used to determine whether the first oxidase such as the polyol oxidase of the invention does not have significant activity on the second substrate.

TABLE 1
SOX Activity using the (ABTS/HRP Assay) with 6
different E. coli cell extracts. P10 extract is
a negative control with background activity.

| | |
|---|---|
| 1. | Assay reagent (100 mM Kpi, pH 7, 1% Sorbitol, 5 mM ABTS, |
| | 10 U7 ml HRP) |
| 2. | 37° C., 990 μl if assay reagent |
| 3. | 3 minutes |
| 4. | P10 control = 0.2 U/mg |
| 5. | P18 = 1.2 U/mg |
| 6. | P6 = 1.05 U/mg |
| 7. | P6H = 1.49 U/mg |
| 8. | P19 = 0.69 U/mg |
| 9. | P19H = 1.17 U/mg |
| 10. | Oda & Hiraga E. coli expression of SOX at cell extract |
| | stage = 1.1 U/mg. |

Table 1 shows the production of sorbitol oxidase as an active enzyme in E. coli BL21(DE3)pLysS. The negative control showed a background activity of 0.2 U/mg. The enzyme activity was improved by heating (H) the cell free extracts P6H & P19H, indicating that a heat treatment step can be used for purification of the SOX protein. The E. coli expressed SOX have specific activity similar to previous work carried out by Kohei Oda & Kazumi Hiraga (Biosci Biotech Biochem 61:1699-1704, 1997).

### Example 3

### Expression of the Putative Sorbitol Oxidase Gene from Streptomyces coelicolor/lividans in Streptomyces lividans Strain S3G3

The Streptomyces coelicolor gi|28380233|sp|Q9ZBU1|XYOA_STRCO annotated as a probable xylitol oxidase (XOX) has been identified by blast searches to be the closest in sequence identity (between 54-60% depending on the alignment) to Streptomyces sp H-7775 sorbitol oxidase gene. The locus containing the Streptomyces coelicolor putative XOX gene, SCO6147, ORFNames=SC1A9.11c sequence was retrieved from the sequence database and several gene specific primers & flanking primers were used to isolate the corresponding S. lividans gene by PCR. The Streptomyces lividans complete genome sequence is not available yet but is almost identical to the fully sequenced S. coelicolor genome. Final PCR reaction consisted of Primers us-sco1 5′gcccatatgagcgacatcacggtcacc (SEQ ID NO 9) and Is-sco1 5′ ggatcctcagcccgcgagcacccc (SEQ ID NO 10), genomic DNA from S. lividans as the template resulting in the synthesis of a 1.269 kb PCR fragment. The PCR conditions used in this final PCR step consisted of 30 cycles: denaturation at 94° C. for 55 seconds, annealing at 55° C. for 55 seconds, extension for 1-2 minutes at 68° C. The polymerase used is Platinum Pfx DNA polymerase plus enhancer solution from Invitrogen. The resulting PCR product was cloned directly in an E. coli vector PCR Blunt TOPO. Primers us-s1 5′gccatgggcgacatcacggtcaccaac (SEQ ID NO 11), and Is-s1 5′ atggatcctcagcccgcgagcacccc (SEQ ID NO 12), were used in a PCR reaction using the same conditions as above. The 1.268 kb PCR product was digested with NcoI-BamH1 and the resulting fragment was cloned directly into an Nco1+Bam H1 digested Streptomyces vector pKB105. The final construct is the expression vector designated as PSMM-SOX (S. lividans) in FIG. 6. The cloned SOX in PCR Blunt TOPO was used to verify the nucleotide sequence of the putative SOX gene. The 5 ul of plasmid DNA (FIG. 6) was used to transform Streptomyces g3s3 protoplasts. Transformation reaction was plated on R5 plates and incubated at 32° C. for 18 hours. Soft nutrient agar overlay containing thiostrepton was poured on the plates that were further incubated for 3 days. Single colonies were used to inoculate a 250 ml flask with 20 ml TS-G media containing thiostrepton. After 3 days of cultivation with shaking at 30° C., 2 ml aliquots were used to inoculate a 250 ml flask containing the Production media. The cell pellets was collected by centrifugation, resuspended in buffer and disrupted. Table 2 shows the SOX activities present in the cell-free extracts derived from the different Streptomyces transformants.

TABLE 2
SOX activities of the cell-free extracts from 20 different
Streptomyces transformants showing varying amounts of activity.
Putative SOX Activity Assay (ABTS/HRP) (U/mg).

| Transformant | 17 | 20 | 22 | 24 |
|---|---|---|---|---|
| 1 | 11.38 | 8.03 | 10.89 | 13.60 |
| 2 | 6.77 | 13.14 | 12.38 | 10.38 |
| 3 | 8.88 | 16.81 | 8.85 | 17.03 |
| 4 | 9.37 | 12.32 | 16.30 | 18.28 |
| 5 | 7.34 | 11.68 | 0.02 | 18.14 |

The DNA and protein sequences of the sorbitol oxidase from S. coelicolor (SCO6147) are shown as SEQ ID NO: 2 and 13 respectfully.

### Example 4

### Purification of Sorbitol Oxidase

In this Example, methods used to purify sorbitol oxidase produced by S. lividans (See, Example 1-4), are described. Sorbitol oxidase from Streptomyces lividans is localized in mycelia. Thus, the enzyme was isolated by cell lysis using a French press from cell-extract in 100 mM Kpi buffer, potassium phosphate, pH 7.0. The cell extract was heated to 50° C. for one hour, followed by centrifugation sufficient to remove the debris. The cell lysate supernatant was then mixed with ammonium sulfate to 32% saturation to precipitate the protein fraction containing sorbitol oxidase. The protein precipitate was kept at 4° C. overnight and then was separated from mother liquor by centrifugation 10,000 RPM using Sorvall centrifuge and SLA-1500 Sorvall rotor.

The protein precipitate was then washed with 32% saturated ammonium sulfate solution. The washed protein precipitate was dissolved back in Kpi buffer and the insoluble material was discarded. The soluble fraction was dialyzed against 25 mM Kpi buffer, pH 7.0, overnight and then further purified using affinity chromatography on the reactive orange resin (Prometic). This partially purified sorbitol oxidase preparation and fermentation broth cell lysate (EFT of 108 hrs) were used as samples in experiments for biobleaching. The molecular weight of the enzyme was determined to be ˜45,000 Da. by SDS-PAGE gel electrophoresis. The prosthetic group is a covalently bound FAD (1 mol of FAD to 1 mol of SOX).

### Example 5

### Stability and Bleaching Performance of SOX in HDL Laundry Wash Conditions

In this Example, methods to determine the stability and bleaching performance of SOX in AATCC liquid detergent laundry wash conditions are described. In these experiments, AATCC standard detergent (American Association of Textile Chemists and Colorists Heavy Duty Liquid Detergent Version 2003 without brightener; key components include linear alkane sulfonate, alcohol ethoxylate, propanediol, citric acid, fatty acid, castic soda and water; Testfabrics) is used.

Three bleachable cotton swatches with juice (STC CFT CS-15), wine (STC CFT CS-3), and tea (STC CFT BC-3) are used. The swatches are cut into 15 mm circles with a textile punch (Model B equipped with a ⅝″ die cutter; Model 93046; NAEF).

Single swatch disks are placed into each well of a 24-well microplate (Costar 3526). One (1) ml of washing solution pH 8, containing per liter, 1.5 ml AATCC HDL detergent, 75-100 mM sorbitol, 6 gpg hardness (diluted from stock 15000 gpg hardness solution containing 1.735 M calcium chloride and 0.67 M magnesium chloride), and 0.05% TAED (tetraacetylethylenediamine, Fluka) is added to each well. Five to fifty (5-50 ul) microliters of partially purified sorbitol oxidase or sorbitol oxidase obtained from a late fermentation run (108 hr EFT “effective fermentation time”) produced as described in Examples 1 and 2, is added with a positive displacement pipette to 3-8 wells in one column. Hexose oxidase or glucose oxidase is added at 0.5, 5, 50, 500, 1000 and 1500 U. The control wells contained no enzyme. The microplate was covered with a plastic lid and aluminum foil and incubated at 37° C. with 100 rpm gentle rotation for 14 hr. The plates are then removed from the shaker and tested for the presence of hydrogen peroxide with peroxide test strips (Baker).

One hundred microliters (100 ul) of 0.1 mM sodium carbonate are added to each well to elevate the pH to 10. The microplates are incubated with rotation for another 90 min and the supernatants removed by aspiration. Each well is washed three (3) times with 1.5 ml Dulbecco's PBS, pH 7.3 and three (3) times with 1.5 ml distilled water. Each disk is removed from its well and dried overnight between sheets of paper towels and not exposed to direct light. The disks are inspected visually and then analyzed with a Reflectometer CR-200 (Minolta) calibrated on a standard white tile. The average L values are calculated as the percent soil release (% SR=100%×(Final reflectance−Initial reflectance)/(Reflectance of a white standard-Initial reflectance).

Preliminary results confirm that the sorbitol oxidase/hexose oxidase composition is stable in a typical liquid detergent system and is able to produce effective concentration of hydrogen peroxide in presence of its substrate sorbitol mixed with detergent and available atmospheric oxygen. In addition, the composition generated hydrogen peroxide in presence of a bleach booster (i.e., TAED) is able to help bleach typical colored stains such as blueberry, tea and wine.

### Example 6

### Substrate Range Study of Sorbitol Oxidase

Sorbitol oxidase obtained using the methods described in Example 3-4, was tested for finding its activity with various polyol substrates. All substrates used in the assay were 55 mM in 100 mM phosphate buffer pH 7.0 at 25° C. The relative activity using sorbitol as (++++++=100%) is shown below in Table 1. In addition to these substrates, it is contemplated that other substrates, including, but not limited to glycerol, will find use in the present invention.

| Compound | Relative Activity (+, 0) |
|---|---|
| D-Sorbitol | + + + + + + |
| D-Xylitol | + + + + |
| D-Mannitol | + + + |
| D-Ribitol | + |
| Myo-Inositol | + |
| Glycerol | + |
| 1,3-propanediol | +/2 |
| 1,2-propanediol | +/2 |
| Propylene glycol | 0 |
| Ethylene glycol | 0 |

### Example 7

### Determination of SOX, HOX and GOX Activity

Glucose oxidases have the ability to oxidise glucose to yield hydrogen peroxide. Examples are carbohydrate oxidase, glucooligosaccharide oxidase and glucose oxidase. Hexose oxidase may also be classified as a glucose oxidase, although it typically has a broader specificity, with significant activity on other hexoses asn well as disaccharides, such as maltose.

### Unit Definitions

1 polyol unit (POX) corresponds to the amount of enzyme, which under the specified conditions results in the conversion of 1 μmole of the specified polyol per minute, with resultant generation of 1 μmole of hydrogen peroxide (H₂O₂).

1 sorbitol oxidase (SOX) unit corresponds to the amount of enzyme, which under the specified conditions results in the conversion of 1 μmole sorbitol per minute, with resultant generation of 1 μmole of hydrogen peroxide (H₂O₂).

Definition: 1 hexose oxidase (HOX) unit corresponds to the amount of enzyme which under the specified conditions results in the conversion of 1 μmole of glucose, or alternative hexose sugar, per minute, with resultant generation of 1 μmole of hydrogen peroxide (H₂O₂).

Definition: 1 glucose oxidase (gluOX) unit corresponds to the amount of enzyme which under the specified conditions results in the conversion of 1 μmole of glucose per minute, with resultant generation of 1 μmole of hydrogen peroxide (H₂O₂).

### Assay of SOX, HOX or GOX Activity in Microtiter Plates (300 μl)

The commonly used horse radish peroxidase dye substrate ABTS was incorporated into an assay, measuring the production of H₂O₂ produced by HOX or GOX respectively. ABTS serves as a chromogenic substrate for peroxidase. Peroxidase in combination with H₂O₂ facilitates the electron transport from the chromogenic dye, which is oxidised to an intensely green/blue compound.

### In Vitro Assay

An assay mixture contained 266 μl sorbitol (Sigma P-5504, 0.055 M in 0.1 M sodium phosphate buffer, pH 6.3), (or alternative substrate, e.g. xylitol), 12 μl 2,2′-Azino-bis(3-ethylbenzothiozoline-6-Sulfonic acid) (ABTS) (Sigma A-9941, 5 mg/ml aqueous solution), 12 μl peroxidase (POD) (Sigma P-6782, 0.1 mg/ml in 0.1 M sodium phosphate buffer, pH 6.3) and 10 μl enzyme (SOX or HOX) aqueous solution.

The assay is performed at 25° C.

The incubation was started by the addition of glucose. The absorbance was monitored at 405 nm in an ELISA reader. A standard curve, based on varying concentrations of H₂O₂, was used for calculation of enzyme activity according to the definition above.

The reactions as illustrated using sorbitol can be described in the following manner:

sorbitol+O₂+→glucose+H₂O₂ (1)

glucose+O₂+H₂O→gluconic acid+H₂O₂ (2)

H₂O₂+2ABTS(colorless)+2H⁺→2H₂O+2ABTS(blue/green) (3)

Reaction (1) is catalysed by enzyme (SOX)
Reaction (2) is catalysed by enzyme (HOX or GOX)
Reaction (3) is catalysed by enzyme (POD)

With respect to reaction (2) GOOX and MnCO are also enzymes capable of catalysing this reaction

For ‘in situ’ assays it may be necessary to dilute the matrix prior to performing the assay, so that the effect of interfering substances reacting unspecifically with the assay components become small enough to neglect. Alternatively, the matrix composition may be prepared excluding the interfering substance(s).

### Example 8

### Relative Activity on Xylitol and Sorbitol

The purpose was to measure the difference in activity of sorbitol oxidase on D-sorbitol and D-xylitol. The ABTS assay was used as described previously to measure the rate of H₂O₂ production when both D-sorbitol and D-xylitol was in excess. The same amount of enzyme, as prepared in the previous examples was used in both cases. The table below shows the relative activity on the two substrates (given in percentage values). The enzyme used was as prepared in Examples 3 & 4.

| Substrate | Relative activity% |
|---|---|
| D-sorbitol | 100 |
| D-xylitol | 47.6 |

### Example 9

### Synergy Between the Two Enzymes

The Three Enzymes Used in this Example were:
1. Hexose oxidase: Hoxypure™, available from Danisco A/S.
2. The S. coelicolor (SCO6147) sorbitol oxidase was obtained as described in the previous examples 3-4.
3. Glucose oxidase: Sigma G7141

In the example below the amounts of enzyme are given as unit amounts.

A unit of HOX is the amount of enzyme that produced 1 umol H₂0₂/min when substrates are in excess.

A unit of GOX is the amount of enzyme that produced 1 umol H₂0₂/min when substrates are in excess.

A unit of SOX is the amount of enzyme that produced 1 umol H₂0₂/min when substrates are in excess.

Note. In the examples below, only SOX will have excess substrates. The generated sugar will be the limiting factor for the secondary enzyme. As noted above the activity is given as a percentage value of SOX catalysing D-sorbitol when both D-sorbitol and oxygen is in excess.

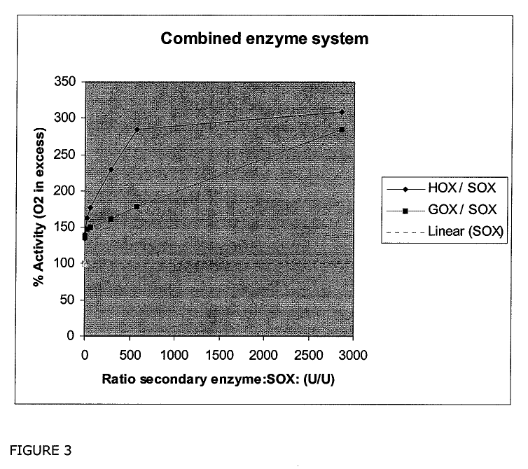

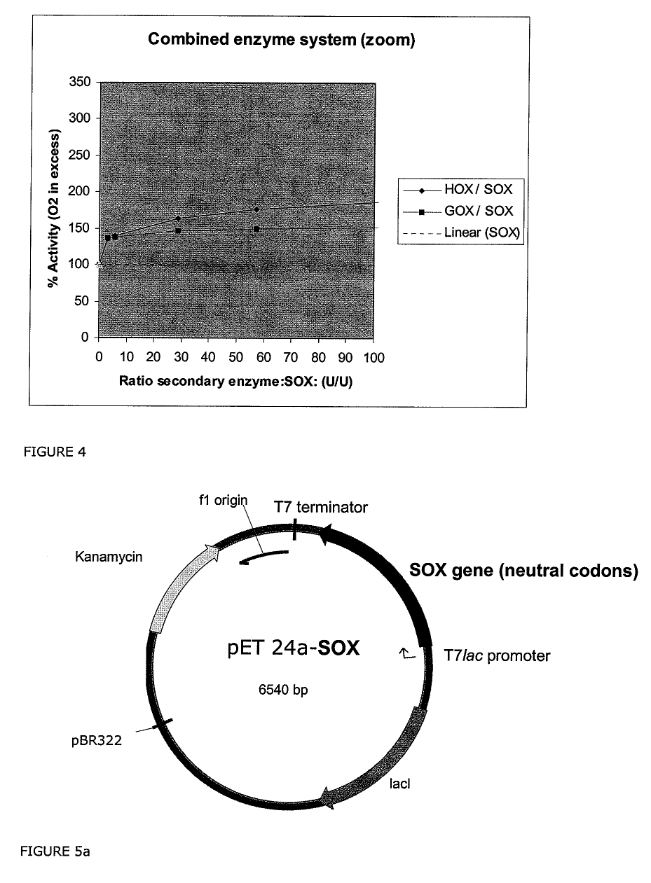

Initial velocities were measured over 5 minutes in 300 uL ABTS assay (as described previously). The production of rate of hydrogen peroxide production was extrapolated from a standard curve. The measured activity, shown in FIG. 3 and FIG. 4 are given as a percentage value. 100% is defined as the rate of hydrogen peroxide production by sorbitol oxidase alone, when the substrates D-sorbitol and oxygen is in excess (Linear velocity curves)

### Example 10

### Chewable Tablet

The enzyme composition is prepared by addition of 1 unit of SOX (as prepared in Examples 1-4), and either hexose oxidase or glucose oxidase is added at 0.5, 5, 50, 500, 1000 and 1500 U to per microliter 100 mM sodium phosphate buffer, pH 6.7, 50 mM sorbitol. The aqueous enzyme composition is used to prepare a chewable tablet. An enzyme composition containing tablet and gum compositions are prepared using conventional base ingredients as set forth below (ingredients listed in terms of wt %).

An enzyme composition containing tablet and gum compositions are prepared using conventional base ingredients as set forth below (ingredients listed in terms of wt %).

| Enzyme composition | 0.5% (provided as part of the water |
|---|---|
| | component) |
| Lycasin 75% | 48.9% |
| Isomalt or xylitol | 23.1% |
| Hydrogenated vegetable oil | 8.7% |
| Water | 4.8% |
| Gelatin (40% solution) | 2.9% |
| Starch coated dicalcium | 8.7% |
| phosphate | |
| Mono-diglyceride mixture | 0.8% |
| Lecithin | 0.3% |
| Aspartame | 0.05% |
| Aspartame K | 0.05% |
| Vanillin | 0.05% |
| Glycerin | 0.1% |
| Sodium bicarbonate | 0.10% |
| Mint flavor | 0.19% |

The chewable tablet is prepared by boiling the Isomalt, Lycasin, water, fat, mono and diglyceride mixture, glycerin, and lecithin to 131° C. after which glycerin is added and the mixture and cooled to 30° C. (HOX) or 60° C. (GOX). Thereafter sodium bicarbonate, the enzyme composition, dicalcium phosphate and the remaining ingredients are added. Thereafter the mixture cooled to room temperature (23° C.) was ground into powder and compressed into a tablet using a tablet press.

In Vivo Plaque Reduction Efficacy The chewable tablet is tested for plaque reduction at 2- and 5-hours after chewing by human volunteers using plaque grown in vivo in an inkra-oral retainer on hydroxyapatite disks. Confocal microscopy is used to visualize and quantify the changes in plaque coverage and plaque ultraskucture. Plaque removal was also measured by conventional light microscopy by staining the plaque before and after treatment with crystal violet indicator and measuring the changes in color intensity. Image Pro Analysis So aware is used to perform the image analysis and the quantitative measurements. The color intensity was measured and used to determine stain removal. The greater the intensity′ the greater the cleaning efficacy.

### Example 11

### Chewing Gum

The following ingredients may be combined to prepare a chewing gum comprising the composition of the invention:

| | |
|---|---|
| Gum base | 31.20% |
| Sorbitol | 28.08% |
| xylitol | 5.23% |
| Enzyme composition | 1.00% (see previous example) |
| Acesulfame K | 0.16% |
| Aspartame | 0.16% |
| Menthol powder | 1.00% |
| Liquid flavor | 0.47% |
| Isomalt PF | 11.70% |
| Isomalt DC | 16.00% |
| Anticaking agents* | 4.00% |
| Flavor | 2.00% |

*Magnesium stearate, talc, silica gel.

### Example 12

### Waterbased Paint

The following ingredients may be combined to prepare a waterbased paint comprising the composition of the invention:

| | |
|---|---|
| Enzyme composition | 0.5% |
| Sorbitol | 0.5% |
| Pigment | 15% |
| Acrylic binder | 25% |
| Water | 50% |
| Other additives* | 9.5% |

*may include the following depending on the type of paint: dispersion agent, suspension agent, dispersant, defoamer, wetting agent, coalescing agent, various fillers, surfactant, co-biocides, thickener, co-preservatives, latex,

### Example 13

### Oilbased Paint

The following ingredients may be combined to prepare a oil based paint comprising the composition of the invention:

| Enzyme composition | 0.5%, dissolved in 80% sorbitol |
|---|---|
| TiO₂ Pigment | 20% |
| Alkyd binder | 40% |
| Hydrocarbon solvent | 20% |
| other additives* | 19.5% |

*may include the following depending on the type of paint: dispersion agent, suspension agent, dispersant, defoamer, wetting agent, coalescing agent, various fillers, surfactant, co-biocides, thickener, co-preservatives, latex,

1. A composition comprising a first oxidase, a first substrate, and at least one further enzyme, wherein, the first substrate is oxidisable by the first oxidase to form hydrogen peroxide and a second substrate; and the second substrate is convertable by the at least one further enzyme to form a product.

2. The composition according to claim 1, wherein the first substrate is a polyol.

3. The composition according to claim 2, wherein the polyol is a pentitol or a hexitol.

4. The composition according to claim 2, wherein the polyol is selected from the group consisting of hexitol, pentitol, sorbitol, xylitol, maltitol, mannitol, galactitol, isomalt, lactitol, arabitol, erythritol, and ribitol.

5. The composition according to claim 4, wherein the polyol is xylitol or sorbitol.

6. (canceled)

7. The composition according to claim 1 wherein the second substrate is a sugar.

8. The composition according to claim 7, wherein the sugar is a monosaccharide or a disaccharide.

9. The composition according to claim 8, wherein the sugar is selected from the group consisting of glucose, xylose, maltose, mannose, galactose, isomaltose, lactose, arabinose, erythrose and ribose.

10. (canceled)

11. The composition according to claim 1, wherein the first oxidase is a polyol oxidase.

12. The composition according to claim 11, wherein the first oxidase is selected from the group consisting of hexitol oxidase, pentitol oxidase, sorbitol oxidase, xylitol oxidase, maltitol oxidase, mannitol oxidase, galactitol oxidase, isomalt oxidase, lactitol oxidase, arabitol oxidase, erythritol oxidase, and ribitol oxidase.

13. The composition according to claims 11, wherein the first oxidase has a polyol oxidase (specific) activity of at least 5 units/g protein when using the respective polyol substrate.

14. The composition according to claim 11, wherein the polyol oxidase is sorbitol oxidase or xylitol oxidase.

15. (canceled)

16. (canceled)

17. The composition according to claim 11, wherein the polyol oxidase has a higher specific activity on sorbitol as compared to xylitol.

18. (canceled)

19. (canceled)

20. (canceled)

21. (canceled)

22. The composition according to claim 1, wherein the at least one further enzyme is a further oxidoreductase.

23. The composition according to claim 22, wherein the second substrate is oxidisable by the further oxidoreductase to form hydrogen peroxide and said product.

24. The composition according to claim 22, wherein the further oxidoreductase is a sugar oxidase.

25. The composition according to claim 23, wherein the sugar-oxidase is selected from the group consisting of: carbohydrate oxidase, oligosaccharide oxidase, maltose oxidase, hexose oxidase, glucose oxidase, mannose oxidase, galactose oxidase, isomaltulose oxidase, lactose oxidase, arabinose oxidase, erythrose oxidase, pentose oxidase, xylose oxidase, and triose oxidase.

26. (canceled)

27. The composition according to claim 22, wherein the amount of the at least one further oxidoreductase compared to the amount of the first oxidase such as the polyol oxidase present in said composition is greater than 1, as measured by the respective number of enzyme units present in said composition.

28. The composition according to claim 1, wherein the presence of the product produced from the second substrate does not reduce the rate of hydrogen peroxide production from the oxidation of the first substrate by the first oxidase.

29. (canceled)

30. The composition according to claim 1, wherein the composition is edible.

31. (canceled)

32. A composition according to claim 1 for use as a medicament.

33. An oral care product comprising a composition according to claim 1 and at least one further ingredient used in oral care products.

34. An oral care product according to claim 33, wherein the oral care product is in the form selected from the group consisting of: chewing gum, a mouthwash, a mouthspray, a pastille, a lozenge, a mouth freshener, a nasal spray, and an oral paste.

35. (canceled)

36. A cosmetic product comprising the composition according to claim 1 and at least one further ingredient used in cosmetic products

37. A skin, hair or teeth bleaching product comprising the composition according to claim 1 and at least one further ingredient used in skin, hair or teeth bleaching and/or whitening products.

38. (canceled)

39. (canceled)

40. A detergent or bleaching product, suitable for use on non-living tissue, comprising the composition according to claim 1, and at least one further ingredient used in detergent or bleaching products.

41. A paint product, such as a maritime, decorative or protective paint, which comprises the composition according to claim 1, and at least one further ingredient used in paint.

42. A pesticide which comprises the composition according to claim 1, and at least one further ingredient used in pesticides.

43. (canceled)

44. (canceled)

45. (canceled)

46. (canceled)

47. (canceled)

48. (canceled)

49. (canceled)

50. A method for the preparation of a composition comprising admixing a first oxidase as defined in claim 1, and a first substrate according to claim 1, and at least one further oxidoreductase according to any one of the preceding claims, and a suitable matrix.

51. (canceled)

52. (canceled)

53. (canceled)

54. (canceled)

55. (canceled)

56. (canceled)

57. A method of medical treatment comprising administering the composition according to claim 1, or an oral care product comprising a composition according to claim 1 and at least one further ingredient used in oral care products, to a patient in need of treatment or prophylaxis.

58. The method according to claim 57, wherein the method is for the treatment or prevention of a medical disorder selected from: gum disease, gingivitis, irritable bowel syndrome, lactose intolerance, colon cancer, high blood cholesterol, high blood pressure, hypertension, infection, inflammation and nutritional deficiencies.

59. A packaged product comprising the composition according to claim 1, wherein said composition is maintained in a oxygen limited environment within said packaged product so as to prevent or reduce the generation of hydrogen peroxide within said packaged product.

60. A kit of parts comprising the following components: first enzyme, at least one further enzyme and a first substrate, wherein the first enzyme and the first substrate are isolated from one another.

61. A method of generating hydrogen peroxide, the method comprising admixing a first oxidase, and a first substrate, and at least one further enzyme and a suitable matrix, under conditions suitable for the generation of both hydrogen peroxide and a second substrate from the oxidation of the first substrate due to the activity of the first oxidase, wherein the second substrate is convertable by the at least one further enzyme into a product.

62. The method according to claim 61, wherein the at least one further enzyme is a further oxidoreductase, which converts the second substrate into further hydrogen peroxide and the product.

63. (canceled)

64. (canceled)

65. (canceled)

66. (canceled)

67. (canceled)

68. (canceled)

69. (canceled)

70. (canceled)

71. (canceled)


SEQUENCE LISTING (13 sequences)

SEQ ID NO: 1 - protein, 420 aa, Streptomyces sp. H7775
MTPAEKNWAGNITFGAKRLCVPRSVRELRETVAASGAVRPLGTRHSFNTVADTSGDHVSLAGLPRVVDIDVPGRAVSLSAGLRFGEFAAELHARGLALANLGSLPHISVAGAVATGTHGSGVGNRSLAGAVRALSLVTADGETRTLRRTDEDFAGAVVSLGALGVVTSLELDLVPAFEVRQWVYEDLPEATLAARFDEVMSAAYSVSVFTDWRPGPVGQVWLKQRVGDEGARSVMPAEWLGARLADGPRHPVPGMPAGNCTAQQGVPGPWHERLPHFRMEFTPSNGDELQSEYFVARADAVAAYEALARLRDRIAPVLQVSELRTVAADDLWLSPAHGRDSVAFHFTWVPDAAAVAPVAGAIEEALAPFGARPHWGKVFSTAPEVLRTLYPRYADFEELVGRHDPEGTFRNAFLDRYFRR

SEQ ID NO: 2 - protein, 418 aa, Streptomyces coelicolor
MGDITVTNWAGNITYTAKELLRPHSLDALRALVADSARVRVLGSGHSFNEIAEPGDGGVLLSLAGLPSVVDVDTAARTVRVGGGVRYAELARVVHARGLALPNMASLPHISVAGSVATGTHGSGVGNGSLASVVREVELVTADGSTVVIARGDERFGGAVTSLGALGVVTSLTLDLEPAYEMEQHVFTELPLAGLDPATFETVMAAAYSVSLFTDWRAPGFRQVWLKRRTDRPLDGFPYAAPAAEKMHPVPGMPAVNCTEQFGVPGPWHERLPHFRAEFTPSSGAELQSEYLMPREHALAALHAMDAIRETLAPVLQTCEIRTVAADAQWLSPAYGRDTVAAHFTWVEDTAAVLPVVRRLEEALVPFAARPHWGKVFTVPAGELRALYPRLADFGALAGALDPAGKFTNAFVRGVLAG

SEQ ID NO: 3 - protein, 47 aa, Streptomyces coelicolor
MTEVSRRKLMKGAAVSGGALALPALGAPPATAAPAAGPEDLPGPAAA

SEQ ID NO: 4 - protein, 468 aa, Streptomyces sp. H7775
MGTEVSRRKLMKGAAVSGGALALPALGAPPATAAPAAGPEDLPGPAAAMTPAEKNWAGNITFGAKRLCVPRSVRELRETVAASGAVRPLGTRHSFNTVADTSGDHVSLAGLPRVVDIDVPGRAVSLSAGLRFGEFAAELHARGLALANLGSLPHISVAGAVATGTHGSGVGNRSLAGAVRALSLVTADGETRTLRRTDEDFAGAVVSLGALGVVTSLELDLVPAFEVRQWVYEDLPEATLAARFDEVMSAAYSVSVFTDWRPGPVGQVWLKQRVGDEGARSVMPAEWLGARLADGPRHPVPGMPAGNCTAQQGVPGPWHERLPHFRMEFTPSNGDELQSEYFVARADAVAAYEALARLRDRIAPVLQVSELRTVAADDLWLSPAHGRDSVAFHFTWVPDAAAVAPVAGAIEEALAPFGARPHWGKVFSTAPEVLRTLYPRYADFEELVGRHDPEGTFRNAFLDRYFRR

SEQ ID NO: 5 - DNA, 1415 bp, Artificial Sequence
  Nucleotide seqeunce of fusion between signal seqeunce and SEQ ID No 1
ccatgggcaccgaggtctcccgccgcaagctgatgaagggcgcggcggtgtcgggcggcgcgctggcgctgccggccctcggcgccccgcccgccaccgcggcgccggccgccggccccgaggacctcccgggccccgccgccgccatgaccccggccgagaagaactgggccggcaacatcaccttcggcgccaagcgcctgtgcgtcccgcgctccgtccgcgagctgcgcgagaccgtggccgcctccggcgccgtgcgcccgctgggcacccgccactcgttcaacaccgtcgccgacacctccggcgaccacgtgtcgctggccggcctgccgcgcgtcgtcgacatcgacgtcccgggccgggccgtgtccctgtccgccggcctgcgcttcggcgagttcgccgccgagctgcacgcccgcggcctggccctggccaacctgggctccctgccgcacatctccgtggcgggcgcggtcgccaccggcacccacggctccggcgtcggcaaccgctccctggcgggcgccgtccgcgccctgtccctggtcaccgccgacggcgagacccgcaccctgcgccgcaccgacgaggacttcgccggcgccgtcgtgtccctgggcgccctgggcgtcgtcacctccctggagctggacctggtcccggccttcgaggtccgccagtgggtctacgaggacctgcccgaggccaccctggccgcccgcttcgacgaggtcatgtccgccgcctactccgtgtccgtgttcaccgactggcgcccgggcccggtcggccaggtctggctgaagcagcgcgtcggcgacgagggcgcccgctccgtcatgccggccgagtggctgggcgcccgcctggccgacggcccgcgccacccggtccccggcatgcccgccggcaactgcaccgcccagcagggcgtcccgggcccgtggcacgagcgcctgccgcacttccgcatggagttcaccccgtccaacggcgacgagctgcagtccgagtacttcgtcgcccgcgcggacgccgtcgcggcctacgaggcgctggcccgcctgcgcgaccgcatcgccccggtcctgcaggtctccgagctgcgcaccgtcgccgccgacgacctgtggctgtccccggcccacggccgcgactccgtcgccttccacttcacctgggtcccggacgccgccgccgtcgccccggtcgccggcgccatcgaggaggccctggccccgttcggcgcccgcccgcactggggcaaggtgttctccaccgcccccgaggtcctgcgcaccctgtacccgcgctacgccgacttcgaggagctggtcggccgccacgaccccgagggcaccttccgcaacgccttcctcgaccgctacttccgccgctgaggatcc

SEQ ID NO: 6 - DNA, 52 bp, Artificial Sequence
  PCR primer
gcgctagccggccccccggcacaggccatgaccccggccgagaagaactggg

SEQ ID NO: 7 - DNA, 17 bp, Artificial Sequence
  PCR primer
caggaaacagctatgac

SEQ ID NO: 8 - DNA, 1370 bp, Artificial Sequence
  Nucleotide sequence encoding fusion between signal seqeunce and SEQ ID No 1 with introduced RE sites
gcgctagccggccccccggcacaggccatgaccccggccgagaagaactgggccggcaacatcaccttcggcgccaagcgcctgtgcgtcccgcgctccgtccgcgagctgcgcgagaccgtggccgcctccggcgccgtgcgcccgctgggcacccgccactcgttcaacaccgtcgccgacacctccggcgaccacgtgtcgctggccggcctgccgcgcgtcgtcgacatcgacgtcccgggccgggccgtgtccctgtccgccggcctgcgcttcggcgagttcgccgccgagctgcacgcccgcggcctggccctggccaacctgggctccctgccgcacatctccgtggcgggcgcggtcgccaccggcacccacggctccggcgtcggcaaccgctccctggcgggcgccgtccgcgccctgtccctggtcaccgccgacggcgagacccgcaccctgcgccgcaccgacgaggacttcgccggcgccgtcgtgtccctgggcgccctgggcgtcgtcacctccctggagctggacctggtcccggccttcgaggtccgccagtgggtctacgaggacctgcccgaggccaccctggccgcccgcttcgacgaggtcatgtccgccgcctactccgtgtccgtgttcaccgactggcgcccgggcccggtcggccaggtctggctgaagcagcgcgtcggcgacgagggcgcccgctccgtcatgccggccgagtggctgggcgcccgcctggccgacggcccgcgccacccggtccccggcatgcccgccggcaactgcaccgcccagcagggcgtcccgggcccgtggcacgagcgcctgccgcacttccgcatggagttcaccccgtccaacggcgacgagctgcagtccgagtacttcgtcgcccgcgcggacgccgtcgcggcctacgaggcgctggcccgcctgcgcgaccgcatcgccccggtcctgcaggtctccgagctgcgcaccgtcgccgccgacgacctgtggctgtccccggcccacggccgcgactccgtcgccttccacttcacctgggtcccggacgccgccgccgtcgccccggtcgccggcgccatcgaggaggccctggccccgttcggcgcccgcccgcactggggcaaggtgttctccaccgcccccgaggtcctgcgcaccctgtacccgcgctacgccgacttcgaggagctggtcggccgccacgaccccgagggcaccttccgcaacgccttcctcgaccgctacttccgccgctgaggatccgagctccagcttttgttccctttagtgagggttaattgcgcgcttggcgtaatcatggtcatagctgtttcctg

SEQ ID NO: 9 - DNA, 27 bp, Artificial Sequence
  PCR primer
gcccatatgagcgacatcacggtcacc

SEQ ID NO: 10 - DNA, 24 bp, Artificial Sequence
  PCR primer
ggatcctcagcccgcgagcacccc

SEQ ID NO: 11 - DNA, 27 bp, Artificial Sequence
  PCR primer
gccatgggcgacatcacggtcaccaac

SEQ ID NO: 12 - DNA, 26 bp, Artificial Sequence
  PCR primer
atggatcctcagcccgcgagcacccc

SEQ ID NO: 13 - DNA, 1268 bp, Artificial Sequence
  Streptomyces lividans and Streptomyces coelicolor SOX with the addition of the NCO1 cloning site at the start methionine
gccatgggcgacatcacggtcaccaactgggccggcaacatcacgtacacggcgaaggaactgctgcggccgcactccctggacgcgctgcgggccctggtggcggacagcgccagggtgcgggtgctgggcagcgggcactccttcaacgagatcgccgagccgggcgacgggggtgtcctgctgtcgctggcgggcctgccgtccgtggtggacgtggacacggcggcccgtacggtgcgggtcggcggcggtgtgcggtacgcggagctggcccgggtggtgcacgcgcggggcctggcgctgccgaacatggcctcgctgccgcacatctcggtcgccgggtcggtggccaccggcacccacggttcgggggtgggcaacggttcgctggcctcggtggtgcgcgaggtggagctggtcaccgcggacggttcgaccgtggtgatcgcgcggggcgacgagcggttcggcggggcggtgacctcgctcggcgcgctgggcgtggtgacgtcgctcacactcgacctggagccggcgtacgagatggaacagcacgtcttcaccgagctgccgctggccgggttggacccggcgacgttcgagacggtgatggcggcggcgtacagcgtgagtctgttcaccgactggcgggcgcccggtttccggcaggtgtggctgaagcggcgcaccgaccggccgctggacggtttcccgtacgcggccccggccgccgagaagatgcatccggtgccgggcatgcccgcggtgaactgcacggagcagttcggggtgccggggccctggcacgagcggctgccgcacttccgcgcggagttcacgcccagcagcggtgccgagttgcagtcggagtacctgatgccccgggagcacgccctggccgccctgcacgcgatggacgcgatacgggagacgctcgcgccggtgctccagacctgcgagatccgcacggtcgccgccgacgcgcagtggctgagcccggcgtacgggcgggacaccgtggccgcgcacttcacctgggtcgaggacacggcggcggtgctgccggtggtgcggcggctggaggaggcgctcgtccccttcgcggcccgtccgcactgggggaaggtgttcaccgtcccggcgggcgagctgcgtgcgctgtacccgcggctggccgacttcggggcgctggccggggcgctggacccggcggggaagttcaccaacgcgttcgtgcgcggggtgctcgcgggctgaggatccat